(19) United States
(12) Patent Application Publication
(10) Pub. No.: US 2021/0170168 A1
(43) Pub. Date: Jun. 10, 2021

(54) IMPLANTABLE BIOELECTRIC DEVICES AND METHODS OF USE

(71) Applicant: Vomaris Innovations, Inc., Tempe, AZ (US)

(72) Inventors: Michael Nagel, Tempe, AZ (US); Mary Maijer, Tempe, AZ (US); Charmaine Sutton, Tempe, AZ (US); Wendell King, Tempe, AZ (US)

(21) Appl. No.: 16/770,896

(22) PCT Filed: Dec. 7, 2018
(86) PCT No.: PCT/US2018/064487

§ 371 Date: Jun. 8, 2020

Related U.S. Application Data
(60) Provisional application No. 62/596,502, filed on Dec. 8, 2017.
(60) Provisional application No. 62/721,305, filed on Aug. 22, 2018.

(51) Int. Cl. A61N 1/32; A61N 1/04; A61N 1/05; A61N 1/378

(52) U.S. Cl. CPC A61N 1/326; A61N 1/0468; A61N 1/378; A61N 1/0504; A61N 1/0492

(57) ABSTRACT

A resorbable bioelectric device includes multiple first reservoirs and multiple second reservoirs joined with a planar substrate. Selected ones of the multiple first reservoirs include a reducing agent, and first reservoir surfaces of selected ones of the multiple first reservoirs are proximate to a first substrate surface. Selected ones of the multiple second reservoirs include an oxidizing agent, and second reservoir surfaces of selected ones of the multiple second reservoirs are proximate to the first substrate surface.

### FIELD

The present specification relates to implantable bioelectric devices, and methods of manufacture and use thereof.

### BACKGROUND

Biologic tissues and cells are affected by electrical stimulus. The present Specification relates to systems, methods and devices useful for applying electric fields and/or currents to a treatment area.

### SUMMARY

Disclosed herein are systems, devices, and methods for use in treatment of subjects. In embodiments the system or device comprises a bio-resorbable (“resorbable”) material, for example a resorbable polymeric material which breaks down over time, comprising one or more biocompatible electrodes configured to generate at least one of a low level electric field (LLEF) or low level electric current (LLEC). Embodiments disclosed herein can produce a uniform current or field density. By making the disclosed systems and devices from a resorbable material, for example one with controlled degradation rate, the material may be resorbed by their in situ environment over a predetermined time period. For instance, a resorbable material can be suitable for being resorbed within a body, for example, a mammal such as a human or animal. Consequently, a temporary medical device made from a resorbable material can be left in the body to be resorbed over time. During resorption of the temporary medical device, specific and/or therapeutic agents can be released in a controlled manner.

Disclosed embodiments comprise biodegradable and resorbable polymer pouches, for example for use in enclosing, completely or partially, implantable medical devices (IMDs), such as cardiac rhythm management devices (CRMs), i.e., a pouch, covering, or other receptacle capable of encasing, surrounding and/or holding the CRM or other IMD for the purpose of securing it in position, inhibiting or reducing bacterial growth, providing pain relief and/or inhibiting scarring or fibrosis on or around the CRM or other IMD. In embodiments, the biodegradable and resorbable pouches disclosed herein include one or more drugs in the polymer matrix to provide prophylactic effects and alleviate side effects or complications associated with the surgery or implantation of the CRM or other IMD. In embodiments, the polymer matrix of the fully resorbable pouches can comprise one or more drugs. Such drugs include, but are not limited to, antimicrobial agents, anti-fibrotic agents, anesthetics and anti-inflammatory agents as well as other classes of drugs, including biological agents such as proteins, growth inhibitors and the like. In embodiments, a pouch can be formed at the time of use from a “tape” comprising one or more biocompatible electrodes configured to generate at least one of a low level electric field (LLEF) or low level electric current (LLEC)

Disclosed embodiments can be fashioned into various sizes and shapes to match the implanted pacemakers, pulse generators, other CRMs and other implantable devices.

The present Specification also discloses bioelectric devices for use in breast reconstruction or augmentation. Breast reconstruction is the rebuilding of a breast, usually in women. It involves using autologous tissue or prosthetic material to construct a natural-looking breast. Breast augmentation involves using breast implants or fat transfer to increase the size of the breasts. In disclosed embodiments the system or device comprises an implant, for example a breast implant, comprising one or more biocompatible electrodes configured to generate at least one of a low level electric field (LLEF) or low level electric current (LLEC). Embodiments disclosed herein can produce a uniform current or field density.

Aspects disclosed herein comprise bioelectric devices that can comprise a multi-array matrix on a substrate, for example a resorbable substrate. Such matrices can include a first array comprising a pattern of microcells formed from a first conductive solution, the first solution comprising a metal species; and a second array comprising a pattern of microcells formed from a second conductive solution, the second solution comprising a metal species capable of defining at least one voltaic cell for spontaneously generating at least one electrical current with the metal species of the first array when said first and second arrays are introduced to an electrolytic solution and said first and second arrays are not in physical contact with each other. Certain aspects utilize an external power source such as AC or DC power or pulsed RF or pulsed current, such as high voltage pulsed current. In one embodiment, the electrical energy is derived from the dissimilar metals creating a battery at each cell/cell interface, whereas those embodiments with an external power source can require conductive electrodes in a spaced apart configuration to predetermine the electric field shape and strength.

Systems and devices disclosed herein can comprise corresponding or interlocking perimeter areas or substrate shapes to assist the devices in maintaining their position on or in the patient and/or their position relative to each other.

Disclosed embodiments comprise methods and systems for applying the disclosed systems and devices. Further disclosed herein are adjustable systems, devices, and methods for use in treatment of subjects, in particular treatment of specific areas of tissue where a resorbable dressing is preferred. Disclosed embodiments comprise devices that can be applied in a user-determined configuration to provide effective treatment in a variety of positions and lengths. For example, embodiments can be applied in user-determined lengths and angles, for example to “wrap” surgical devices of various types.

Embodiments can be used to treat tissue by activating enzymes, increasing and directing cellular migration, increasing glucose uptake, driving redox signaling, increasing H₂O₂ production, increasing cellular protein sulfhydryl levels, and increasing (IGF)-1 R phosphorylation. Embodiments can also up-regulate integrin production and accumulation in treatment areas.

Disclosed embodiments comprise methods and devices for prevention of biofilms. Disclosed embodiments comprise methods and devices for treatment of biofilms. Additional aspects include methods and devices for preventing bacterial biofilm formation. Aspects also include methods and devices for reducing microbial or bacterial proliferation, killing microbes or bacteria, killing bacteria through a biofilm layer, and preventing the formation of a biofilm. Embodiments include methods of using devices disclosed herein in combination with antibiotics for reducing microbial or bacterial proliferation, killing microbes or bacteria, killing bacteria through a biofilm layer, or preventing the formation of a biofilm.

Certain embodiments comprise a solution or formulation comprising an active agent and a solvent or carrier or vehicle.

### BRIEF DESCRIPTION OF THE DRAWINGS

FIG. 1 is a detailed plan view of the electrode array of an embodiment disclosed herein.

FIG. 2 is a detailed plan view of a pattern of applied electrical conductors in accordance with an embodiment disclosed herein.

FIG. 3 is a detailed plan view of the electrode array of an alternate embodiment disclosed herein which includes fine lines of conductive metal solution connecting electrodes.

FIG. 4 is a detailed plan view of the electrode array of another alternate embodiment having a line pattern and dot pattern.

FIG. 5 is a detailed plan view of the electrode array of yet another alternate embodiment having two line patterns.

FIG. 6 is a pacemaker suitable for use with disclosed embodiments.

FIG. 7 depicts a disclosed “folding” pouch embodiment enclosing a pacemaker.

FIG. 8 depicts a disclosed pouch embodiment enclosing a pacemaker.

FIG. 9 depicts a disclosed pouch embodiment enclosing a pacemaker.

FIG. 10 depicts a disclosed pouch embodiment.

FIG. 11 depicts a disclosed pouch embodiment.

FIG. 12 depicts a disclosed pouch embodiment enclosing a pacemaker.

FIGS. 13A and 13B depict exemplary embodiments showing the electrode array on a breast implant surface.

### DETAILED DESCRIPTION

Embodiments disclosed herein comprise methods, systems and devices that can provide a low level electric field to a treatment area or, when brought into contact with an electrically conducting material, can provide a low level electric current to a treatment area. Thus, in embodiments an LLEC system is an LLEF system that is in contact with an electrically conducting material, for example a liquid material. In certain embodiments, the micro-current or electric field can be modulated, for example, to alter the duration, size, shape, field depth, duration, current, polarity, or voltage of the system. For example, it can be desirable to employ an electric field of greater strength or depth in a particular treatment area to achieve optimal treatment. In embodiments the watt-density of the system can be modulated.

In embodiments the size, shape, or both, of the disclosed systems and devices can be adjusted, for example by the user or practitioner. In embodiments the system comprises multiple components, for example two or more, that can be applied to create an implant, resorbable dressing, or substrate of the desired volume and shape.

Embodiments disclosed herein include methods of treatment. For example, a method of treatment disclosed herein can comprise applying an embodiment disclosed herein to a tissue, for example, the skin, a muscle or muscle group, a joint, a wound, an incision, or the like. Further embodiments disclosed herein can comprise applying a substrate or tape disclosed herein to surround or wrap an implantable medical device, for example prior to insertion into the body.

Embodiments disclosed herein include methods of use of disclosed devices. For example, a method of use disclosed herein can comprise breast reconstruction or augmentation surgery using a disclosed breast implant comprising a first array comprising a pattern of microcells formed from, for example, a first conductive solution, the first solution comprising a metal species; and a second array comprising a pattern of microcells formed from a second conductive solution, the second solution comprising a metal species capable of defining at least one voltaic cell for spontaneously generating at least one electrical current with the metal species of the first array when said first and second arrays are introduced to an electrolytic solution and said first and second arrays are not in physical contact with each other.

### Definitions

“Activation agent” as used herein means a composition useful for maintaining a moist and/or electrically conductive environment within and about the treatment area. Activation agents can be in the form of gels or liquids. Activation agents can be conductive. Activation agents can provide a temperature increase or decrease to an area where applied. Activation gels can be antibacterial.

“Active agent” as used herein means an ingredient or drug that is biologically active and can be present in a formulation or solution. Disclosed formulations can contain more than one active ingredient.

“Affixing” as used herein can mean contacting a patient or tissue or implant with a device or system disclosed herein. In embodiments “affixing” can comprise the use of straps, elastic, adhesive, etc. With regard to embodiments disclosed herein, “affixing” can comprise the overlapping, sequential application of disclosed devices.

“Antimicrobial agent” as used herein refers to an additional agent that kills or inhibits the growth of microorganisms. One type of antimicrobial agent can be an antibacterial agent. “Antibacterial agent” or “antibacterial” as used herein refers to an agent that interferes with the growth and reproduction of bacteria. Antibacterial agents are used to disinfect surfaces and eliminate potentially harmful bacteria. Unlike antibiotics, they are not used as medicines for humans or animals, but are found in products such as soaps, detergents, health and skincare products and household cleaners.

“Applied” or “apply” as used herein refers to contacting a surface or substrate with a conductive material, for example printing, painting, or spraying a conductive ink on a surface. Alternatively, “applying” can mean contacting a treatment area with a device or system disclosed herein.

“Conductive material” as used herein refers to an object or type of material which permits the flow of electric charges in one or more directions. Conductive materials can comprise solids such as metals or carbon, or liquids such as conductive metal solutions and conductive gels. Conductive materials can be applied to form at least one matrix. Conductive liquids can dry, cure, or harden after application to form a solid material. Solid material can also be cast from a polymer solution that contains conductive material and water wherein the water evaporates when the conductive liquids dry, cure, or harden. Solid material can then be activated when soaked in water for use.

“Discontinuous region” as used herein refers to a “void” in a material such as a hole, slot, or the like. The term can mean any void in the material though typically the void is of a regular shape. A void in the material can be entirely within the perimeter of a material or it can extend to the perimeter of a material.

“Dots” as used herein refers to discrete deposits of similar or dissimilar reservoirs that can, in certain embodiments, function as at least one battery cell. The term can refer to a deposit of any suitable size or shape, such as squares, circles, triangles, lines, etc. The term can be used synonymously with, microcells, microspheres, etc. “Microspheres” refers to small spherical particles, with diameters in the micrometer range (typically 1 μm to 1000 μm [1 mm]). Microspheres are sometimes referred to as microparticles. Microspheres can be manufactured from various natural and synthetic materials. The term can be used synonymously with, micro balloons, beads, particles, etc.

“Electrode” refers to discrete deposits of similar or dissimilar conductive materials. In embodiments utilizing an external power source the electrodes can comprise similar conductive materials. In embodiments that do not use an external power source, the electrodes can comprise dissimilar conductive materials that can define an anode and a cathode.

“Expandable” as used herein refers to the ability to stretch while retaining structural integrity and not tearing. The term can refer to solid regions as well as discontinuous or void regions; solid regions as well as void regions can stretch or expand. “Expandable” can refer to stretching along any axis, including the “Z” axis, that is, wherein the dressing expands away from the treatment site while maintaining contact with the treatment site.

“Interlocking” as used herein refers to areas on the perimeter of disclosed devices that complement other areas on the perimeter such that the areas engage with each other by the fitting together of projections and recesses, for example in a “ball” and “socket” configuration. This design can enable disclosed devices to “nest” closely together to treat multiple areas in close proximity to one another.

“Matrix” or “matrices” as used herein refer to a pattern or patterns, such as those formed by electrodes on a surface, such as a substrate or pouch or fabric or fiber or microparticle, or the like. Matrices can also comprise a pattern or patterns within a solid or liquid material or a three dimensional object. Matrices can be designed to vary the electric field or electric current or microcurrent generated. For example, the strength and shape of the field or current or microcurrent can be altered, or the matrices can be designed to produce an electric field(s) or current or microcurrent of a desired strength or shape.

“Pouch” or “pouches,” means any pouch, bag, skin, shell, covering, or other receptacle formed from a biodegradable polymer or from a any fully resorbable polymer film and shaped to encapsulate, encase, surround, cover or hold, in whole or in substantial part, an implantable medical device. Disclosed pouches can have openings to permit leads and tubes of the IMD to extend unhindered from the IMD though the opening of the pouch. The pouches may also have porosity to accommodate monopolar devices that require the IMD to be electrically grounded to the surrounding tissue. An IMD is substantially encapsulated, encased, surrounded or covered when the pouch can hold the device and at least 10%, 20%, 30%, 50%, 60%, 75%, 80%, 85%, 90%, 95% or 98% of the device is within the pouch or covered by the pouch.

“Reduction-oxidation reaction” or “redox reaction” as used herein refers to a reaction involving the transfer of one or more electrons from a reducing agent to an oxidizing agent. The term “reducing agent” can be defined in some embodiments as a reactant in a redox reaction, which donates electrons to a reduced species. A “reducing agent” is thereby oxidized in the reaction. The term “oxidizing agent” can be defined in some embodiments as a reactant in a redox reaction, which accepts electrons from the oxidized species. An “oxidizing agent” is thereby reduced in the reaction. In various embodiments a redox reaction produced between a first and second reservoir provides a current between the dissimilar reservoirs. The redox reactions can occur spontaneously when a conductive material is brought in proximity to first and second dissimilar reservoirs such that the conductive material provides a medium for electrical communication and/or ionic communication between the first and second dissimilar reservoirs. In other words, in an embodiment electrical currents can be produced between first and second dissimilar reservoirs without the use of an external battery or other power source (e.g., a direct current [DC] such as a battery or an alternating current (AC) power source such as a typical electric outlet). Accordingly, in various embodiments a system is provided which is “electrically self contained,” and yet the system can be activated to produce electrical currents. The term “electrically self contained” can be defined in some embodiments as being capable of producing electricity (e.g., producing current) without an external battery or power source. The term “activated” can be defined in some embodiments to refer to the production of electric current through the application of a radio signal of a given frequency or through ultrasound or through electromagnetic induction.

“Resorbable” as used herein refers to a resorbable material, e.g. an at least partially resorbable material or a fully resorbable material.

“Stretchable” as used herein refers to the ability of embodiments that stretch without losing their structural integrity. That is, embodiments can stretch to accommodate irregular tissue surfaces or surfaces wherein one portion of the surface can move relative to another portion.

“Tape” as used herein includes an adhesive or non-adhesive substrate which includes one or more biocompatible electrodes configured to generate at least one of a low level electric field (LLEF) or low level electric current (LLEC).

“Treatment” as used herein can include the use of disclosed embodiments on or in a patient.

Systems, Devices, and Methods of Manufacture

In embodiments, systems and devices disclosed herein can apply an electric field, an electric current, or both, wherein the field, current, or both can be of varying size, strength, density, shape, or duration in different areas of the embodiment. In embodiments, systems and devices disclosed herein can apply an electric field, an electric current, or both, wherein the field, current, or both can be of uniform size, strength, density, shape, or duration. In embodiments, by micro-sizing the electrodes or reservoirs, the shapes of the electric field, electric current, or both can be customized, increasing or decreasing very localized watt densities and allowing for the design of patterns of electrodes or reservoirs wherein the amount of electric field over a tissue can be designed or produced or adjusted based upon feedback from the tissue or upon an algorithm within sensors operably connected to the embodiment and a control module. The electric field, electric current, or both can be stronger in one zone and weaker in another. The electric field, electric current, or both can change with time and be modulated based on treatment goals or feedback from the tissue or patient. The control module can monitor and adjust the size, strength, density, shape, or duration of electric field or electric current based on material parameters or tissue parameters. For example, embodiments disclosed herein can produce and maintain very localized electrical events. For example, embodiments disclosed herein can produce specific values for the electric field duration, electric field size, electric field shape, field depth, current, polarity, and/or voltage of the device or system.

Embodiments disclosed herein can comprise multiple layers. Embodiments can be ETO and Gamma Sterilization compatible.

Disclosed embodiments can comprise pouches comprising matrices of biocompatible electrodes. The pouches can be fashioned into various sizes and shapes to match the implanted pacemakers, pulse generators, other CRMs and other implantable devices.

Disclosed embodiments can comprise tapes comprising matrices of biocompatible electrodes. The tapes can be fashioned into various sizes and shapes to “wrap” the implanted pacemakers, pulse generators, other CRMs and other implantable devices.

Pouches, tapes, and the like can be made from made from fully resorbable and biodegradable polymers which can be formed into films, molded, electrospun and shaped as desired into pouches, bags, coverings, skins, shells or other receptacle and the like. Pouches of the invention have one or more biodegradable polymers to impart or control drug elution of particular profiles or other temporary effects.

Disclosed pouches can comprise one or more biodegradable polymers, optionally in layers, and each layer independently further containing one or more drugs. The physical, mechanical, chemical, and resorption characteristics of the polymer enhance the clinical performance of the pouch and the surgeon's ability to implant a CRM or other IMD. These characteristics are attained by selecting a suitable thickness for the pouch and one or more biodegradable polymer. In embodiments, it is preferred to use biodegradable polymers with a molecular weight between about 10,000 and about 200,000 Daltons. Such polymers degrade at rates that maintain sufficient mechanical and physical integrity over at least one 1 week at 37° C. in an aqueous environment.

In embodiments the biodegradable polymer comprises a drug, and has a chemical composition complementary to the drug such that the polymer layer can contain between 2-50% drug at room temperature. In one embodiment, the pouch releases drug for at least 2-3 days. Such release is preferred, for example, when the drug is an analgesic to aide in localized pain management at the surgical site. To achieve an analgesic affect, the anesthetic and/or analgesic can be delivered to the injured tissue shortly after surgery or tissue injury. A drug or drugs for inclusion in the pouches of the invention include, but are not limited to analgesics, anti-inflammatory agents, anesthetics, antimicrobial agents, antifungal agents, NSAIDS, other biologics (including proteins and nucleic acids) and the like. Antimicrobial and antifungal agents can prevent the pouch, device, and/or the surrounding tissue from being colonized by bacteria. In embodiments, one or more drugs are incorporated into the polymer matrix that forms the pouches of the invention.

In another embodiment, the pouch coating comprises an anesthetic such that the anesthetic elutes from the implanted pouch to the surrounding tissue of the surgical site for between 1 and 10 days, which typically coincides with the period of acute surgical site pain. In another embodiment, delivery of an antimicrobial drug via a pouch of the invention can create an inhibition zone against bacterial growth and colonization surrounding the implant during the healing process (e.g., usually about 7-30 days or less) and/or prevent undue fibrotic responses.

Anesthetics that contain amines, such as lidocaine and bupivacaine, are hydrophobic and can be difficult to load in sufficient amounts into the most commonly used plastics employed in the medical device industry, such as polypropylene and other non-resorbable thermoplastics. When in their hydrochloride salt form, anesthetics cannot be effectively loaded in significant concentration into such non-resorbable thermoplastics because of the mismatch in hydrophilicity of the two materials. Using biodegradable polymers avoids the issue of drug solubility, impregnation or adherence in or to the underlying device by releasing relatively high, but local, concentrations of those drugs over extended periods of time. For example, by modulating the chemical composition of the biodegradable polymer, a clinically-efficacious amount of anesthetic drug can be incorporated into a pouch of the invention to assure sufficient drug elution and to provide surgical site, post-operative pain relief for the patient. Other elution profiles, with faster or slower drug release over a different (longer or shorter) times, can be achieved by altering the thickness of the film or the layers that form the pouch, the amount of drug in the depot layer and the hydrophilicity of the biodegradable polymer.

Disclosed embodiments can comprise breast implants, for example saline, silicone, or composite breast implants. Saline breast implants are filed with a sterile saline solution. Saline breast implants can comprise structured saline breast implants comprising an inner structure. Silicone breast implants are filled with silicone gel. Silicone breast implants can comprise form-stable breast implants.

Disclosed breast implants con comprise, for example, round breast implants, smooth breast implants, textured breast implants, and combinations thereof.

The implants of the present disclosure can be used for augmentation or reconstruction of the breast. Disclosed embodiments can comprise a variable cohesive gel form stabilizing implant. A variable cohesive gel form stabilizing implant, with shape retention characteristics to maintain form, is characterized by alterations in gel filler cohesiveness. The variable cohesiveness of the gel filler material may be altered by any means (i.e. chemical, fabrication, etc.). It will be appreciated by those skilled in the art that manipulation of the chemical formulation of the gel may result in greater mechanical properties. For example, as more cross-links are formed, a stiffer gel results, allowing for a more form-stable implant.

Substrates

Substrates disclosed herein can comprise cover material, or “shells,” for breast implants, which can comprise flexible materials. For example, substrates suitable for use with disclosed embodiments can include silicone rubber, for example HTV silicone (heat temperature vulcanizing) or RTV silicone (room temperature vulcanizing), elastomeric materials, or the like.

It should be noted that the instant disclosure is not limited to a single shell or envelope. Disclosed embodiments can comprise a single lumen or multiple lumens within its shell, although the use of cohesive gel minimizes the need for separate lumens. The invention may be employed in an implant having either a smooth or textured outer shell. The shell can be circular, oval, crescent-shaped or other suitable shapes. It can be formed of silicone rubber, a laminate of various forms of silicone, silicone copolymers, polyurethane, and various other elastomers in various combinations. The gel cohesiveness may increase with increased volume or dimension of the prosthesis.

Substrates disclosed herein can comprise resorbable materials. For example, suitable resorbable materials can comprise those listed in Table 1:

TABLE 1
Examaples of Additional Biodegradable Polymers for Use
in Construction of the Matrix of this Invention

Aliphatic polyesters
Cellulose
Chitin
Collagen
Copolymers of glycolide
Copolymers of lactide
Elastin
Fibrin
Glycolide/l-lactide copolymers (PGA/PLLA)
Glycolide/trimethylene carbonate copolymers(PGA/TMC)
Hydrogel
Lactide/tetramethylglycolide copolymers
Lactide/trimethylene carbonate copolymers
Lactide/ϵ-caprolactone copolymers
Lactide/σ-valerolactone copolymers
L-lactide/dl-lactide copolymers
Methyl methacrylate-N-vinyl pyrrolidone copolymers
Modified proteins
Nylon-2
PHBA/γ-hydroxyvalerate copolymers (PHBA/HVA)
PLA/Polyethylene oxide copolymers
PLA-polyethylene oxide (PELA)
Poly (amino acids)
Poly (trimethylene carbonates)
Poly hydroxyalkanoate polymers (PHA)
Poly (alklyene oxalates)
Poly (butylene diglycolate)
Poly (hydroxy butyrate) (PHB)
Poly (n-vinyl pyrrolidone)
Poly (ortho esters)
Polyalkyl-2-cyanoacrylates
Polyanhydrides
Polycyanoacrylates
Polydepsipeptides
Polydihydropyrans
Poly-dl-lactide (PDLLA)
Polyesteramides
Polyesters of oxalic acid
Polyglycolide (PGA)
Polyiminocarbonates
Polylactides (PLA)
Poly-l-lactide (PLLA)
Polyorthoesters
Poly-p-dioxanone (PDO)
Polypeptides
Polyphosphazenes
Polysaccharides
Polyurethanes (PU)
Polyvinyl alcohol (PVA)
Poly-β-hydroxypropionate (PHPA)
Poly-β-hydroxybutyrate (PBA)
Poly-σ-valerolactone
Poly-β-alkanoic acids
Poly-β-malic acid (PMLA)
Poly-ϵ-caprolactone (PCL)
Pseudo-Poly (Amino Acids)
Starch
Trimethylene carbonate (TMC)
Tyrosine based polymers

Disclosed embodiments can comprise at least one resorbable matrix, for example a polymeric resorbable matrix. Disclosed resorbable polymer matrices are capable of releasing one or more drugs into surrounding bodily tissue and proximal to the device such that the drug reduces or prevents implant- or surgery-related complications. For example, by including an anesthetic agent, such that the agent predictably seeps or elutes into the surrounding bodily tissue, bodily fluid, or systemic fluid, one has a useful way to attenuate the pain experienced at implantation site. In another example, replacing the anesthetic agent with an anti-inflammatory agent provides a way to reduce the swelling and inflammation associated implantation of the device and/or pouch. In yet another example, by delivering an antimicrobial agent in the same manner and at a therapeutically-effective dose, one has a way to provide a rate of drug release sufficient to prevent colonization of the pouch, the CRM, IMD, or other implant, and/or the surgical implantation site by bacteria for at least the period following surgery necessary for initial healing of the surgical incision.

Thus, the fully resorbable polymer pouches can be formed and shaped to encapsulate, encase or surround a pacemaker, a defibrillator or generator, an implantable access system, a neurostimulator, a drug delivery pump (e.g., intrathecal delivery system or a pain pump) or any other IMD for the purpose of securing those devices in position, providing pain relief, inhibiting scarring or fibrosis and/or for inhibiting bacterial growth on or in the tissue surrounding the device. Films are formed into an appropriate shape to hold the IMD.

In disclosed embodiments, “fully resorbable polymer film” or “films” is used to describe poured films, molded films, sheets, electrospun films, electrospun forms, any form, shape or film made by any other technique, no matter how those entities (i.e., “films”) are made including by pre-forming a shape, injection molding, compression molding, dipping, spraying, electrospinning, thermoforming and the like. The fully resorbable polymer films are made from one or more fully resorbable, biodegradable polymers and are formed into a pouch, covering, skin, shell, receptacle, or other shape suitable for the IMD to encapsulate, encase or otherwise surround and hold, wholly or in substantial part, an IMD. Further, any of these films can be made porous, and the percentage to which they cover the IMD can be adjusted by punching holes, piercing the film (before or after shaping) or forming holes.

The pouches of the invention comprise one or more biodegradable polymers, and optionally contain one or more drugs. Methods of making biodegradable polymers are well known in the art. The biodegradable polymers suitable for use in the invention include but are not limited to polylactic acid, polyglycolic acid and copolymers and mixtures thereof such as poly(L-lactide) (PLLA), poly(D,L-lactide) (PLA); polyglycolic acid [polyglycolide (PGA)], poly(L-lactide-co-D,L-lactide) (PLLA/PLA), poly(L-lactide-co-glycolide) (PLLA/PGA), poly(D, L-lactide-co-glycolide) (PLA/PGA), poly(glycolide-co-trimethylene carbonate) (PGA/PTMC), poly(D,L-lactide-co-caprolactone) (PLA/PCL) and poly(glycolide-co-caprolactone) (PGA/PCL); polyethylene oxide (PEO), polydioxanone (PDS), polypropylene fumarate, poly(ethyl glutamate-co-glutamic acid), poly(tert-butyloxy-carbonylmethyl glutamate), polycaprolactone (PCL), polycaprolactone co-butylacrylate, polyhydroxybutyrate (PHBT) and copolymers of polyhydroxybutyrate, poly(phosphazene), poly(phosphate ester), poly(amino acid), polydepsipeptides, maleic anhydride copolymers, polyiminocarbonates, poly[(97.5% dimethyl-trimethylene carbonate)-co-(2.5% trimethylene carbonate)], poly(orthoesters), tyrosine-derived polyarylates, tyrosine-derived polycarbonates, tyrosine-derived polyiminocarbonates, tyrosine-derived polyphosphonates, polyethylene oxide, polyethylene glycol, polyalkylene oxides, hydroxypropylmethylcellulose, polysaccharides such as hyaluronic acid, chitosan and regenerate cellulose, and proteins such as gelatin and collagen, and mixtures and copolymers thereof, among others as well as PEG derivatives or blends of any of the foregoing.

In some embodiments, biodegradable polymers of the invention comprise diphenol monomer units that are copolymerized with an appropriate chemical moiety to form a polyarylate, a polycarbonate, a polyiminocarbonate, a polyphosphonate or any other polymer. Suitable biodegradable polymers are tyrosine-based polyarylates including those described in U.S. Pat. Nos. 4,980,449; 5,099,060; 5,216,115; 5,317,077; 5,587,507; 5,658,995; 5,670,602; 6,048,521; 6,120,491; 6,319,492; 6,475,477; 6,602,497; 6,852,308; 7,056,493; RE37,160E; and RE37,795E; as well as those described in U.S. Patent Application Publication Nos. 2002/0151668; 2003/0138488; 2003/0216307; 2004/0254334; 2005/0165203; and those described in PCT Publication Nos. WO99/52962; WO 01/49249; WO 01/49311; WO03/091337. These patents and publications also disclose other polymers containing tyrosine-derived diphenol monomer units or other diphenol monomer units, including polyarylates, polycarbonates, polyiminocarbonates, polythiocarbonates, polyphosphonates and polyethers. Likewise, the foregoing patents and publications describe methods for making these polymers, some methods of which may be applicable to synthesizing other biodegradable polymers. Finally, the foregoing patents and publications also describe blends and copolymers with polyalkylene oxides, including polyethylene glycol (PEG). All such polymers are contemplated for use in the present invention.

Representative structures for the foregoing polymers are provided in the above-cited patents and publications which are incorporated herein by reference.

As used herein, DTE is the diphenol monomer desaminotyrosyl-tyrosine ethyl ester; DTBn is the diphenol monomer desaminotyrosyl-tyrosine benzyl ester; DT is the corresponding free acid form, namely desaminotyrosyl-tyrosine. BTE is the diphenol monomer 4-hydroxy benzoic acid-tyrosyl ethyl ester; BT is the corresponding free acid form, namely 4-hydroxy benzoic acid-tyrosine.

P22 is a polyarylate copolymer produced by condensation of DTE with succinate. P22-10, P22-15, P22-20, P22-xx, etc., represents copolymers produced by condensation of (1) a mixture of DTE and DT using the indicated percentage of DT (i.e., 10, 15, 20 and xx % DT, etc.) with (2) succinate.

Additional suitable polyarylates are copolymers of desaminotyrosyl-tyrosine (DT) and an desaminotyrosyl-tyrosyl ester (DT ester), wherein the copolymer comprises from about 0.001% DT to about 80% DT and the ester moiety can be a branched or unbranched alkyl, alkylaryl, or alkylene ether group having up to 18 carbon atoms, any group of which can, optionally have a polyalkylene oxide therein. Similarly, another group of polyarylates are the same as the foregoing but the desaminotyrosyl moiety is replaced by a 4-hydroxybenzoyl moiety. Preferred DT or BT contents include those copolymers with from about 1% to about 30%, from about 5% to about 30% from about 10 to about 30% DT or BT. Preferred diacids (used in forming the polyarylates) include succinate, glutarate, adipate and glycolic acid.

Additional biodegradable polymers useful for disclosed embodiments are the biodegradable, resorbable polyarylates and polycarbonates disclosed in U.S. provisional application Ser. No. 60/733,988, filed Nov. 3, 2005 and in its corresponding PCT Appn. No. PCT/US06/42944, filed Nov. 3, 2006. These polymers, include, but are not limited to, BTE glutarate, DTM glutarate, DT propylamide glutarate, DT glycineamide glutarate, BTE succinate, BTM succinate, BTE succinate PEG, BTM succinate PEG, DTM succinate PEG, DTM succinate, DT N-hydroxysuccinimide succinate, DT glucosamine succinate, DT glucosamine glutarate, DT PEG ester succinate, DT PEG amide succinate, DT PEG ester glutarate and DT PEG ester succinate.

In a disclosed embodiment, the polyarylates are the DTE-DT succinate family of polymers, e.g., the P22-xx family of polymers having from 5-40% DT, including but not limited to, about 1, 2, 5, 10, 15, 20, 25, 27.5, 30, 35 and 40% DT.

Additionally, any of the foregoing polymers used in disclosed embodiments can have from 0.1-99.9% PEG diacid or any other polyalkylene oxide diacid, see e.g., U.S. provisional application Ser. No. 60/733,988, filed Nov. 3, 2005, its corresponding PCT application, filed Nov. 3, 2006 and U.S. Ser. No. 60/983,108, filed Oct. 26, 2007, each of which are incorporated herein by reference. Blends of polyarylates or other biodegradable polymers with polyarylates are also preferred.

The fully resorbable polymer pouches of the invention are prepared using any of the foregoing biodegradable polymers, and preferably using any one or more of the tyrosine-based polyarylates described above. Such polymers are dissolved in appropriate solvents to cast films, prepare spray coating solutions, electrospinning solutions, molding solutions and the like for forming a fully resorbable polymer film of the invention.

Fully resorbable pouches, as films, meshes, non-wovens and the like, can be created by several means: by spray coating a substrate, thermal or solvent casting, weaving, knitting or electrospinning, dip coating, extrusion, or molding. Sheets can be formed into a pouch configuration by thermoforming, or mechanical forming and heat setting, or adhesive, thermal or ultrasonic assembly. Pouches can be constructed directly from resorbable polymer by dip or spray coating a pre-formed shape, or injection or compression molding into the finished shape.

The biodegradable polymers can be formed into multi-layered, fully resorbable polymer films, each layer containing the same or different polymers, the same or different drugs, and the same or different amounts of polymers or drugs. For example, a first film layer can contain drug, while the film layer coating layer contains either no drug or a lower concentration of drug. For example, a multilayer film can be made by casting a first layer, allowing it to dry, and casting a second or successive layer onto the first layer, allowing each layer to dry before casting the next layer.

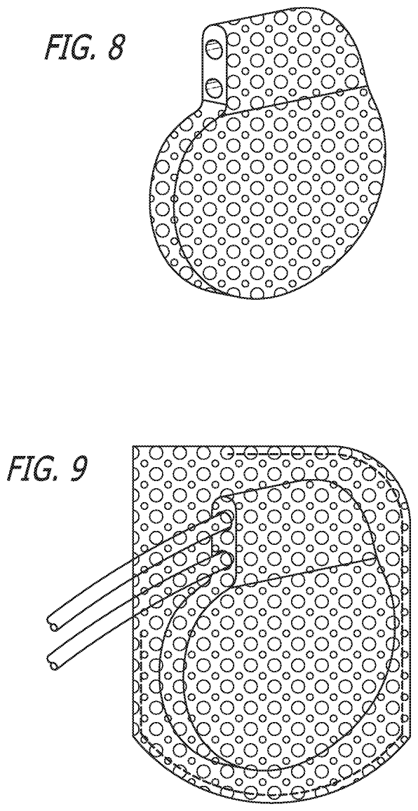

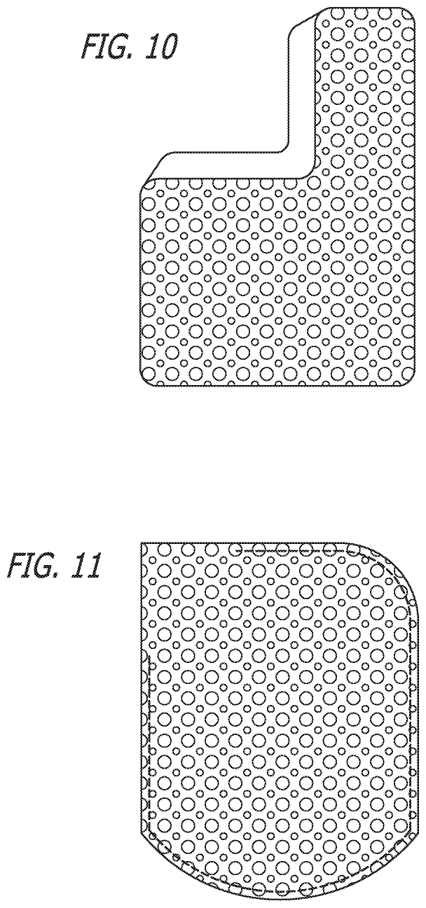

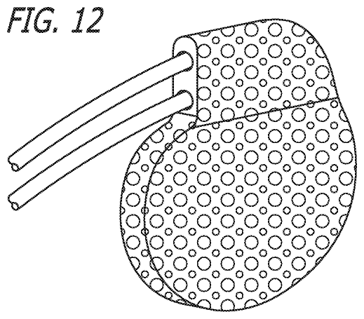

Disclosed pouches can be shaped to fit relatively snugly or more loosely around an IMD. For example, the clamshell shaped pouch shown in FIGS. 8 and 9 is designed to encase the IMD, is capable of being folded, and each half interlocks with the other half to secure the shell around the device and hold the device within the clamshell. Additionally, the clamshell has a space or opening sufficient to allow the leads from the device to pass through the clamshell. The number of spaces or opening in the pouch that are provided can match the number and placement of the leads or other tubes extending from the CRM or other IMD, as applicable for the relevant device. The pouches are constructed with polymers from the group described herein that are selected to have elastomeric properties if is desirable to have a pouch that fits tightly over the IMD.

The films can be laser cut to produce the desired shaped and sized pouches, coverings and the like. Two pieces can be sealed, by heat, by ultrasound or other method known in the art, leaving one side open to permit insertion of the device at the time of the surgical procedure.

In preferred embodiments, the shape and size of the pouch of the invention is similar to that of the DRM or IMD with which it is being used, and the pouch as a sufficient number of openings or spaces to accommodate the leads or tubing of the particular CRM or other IMD.

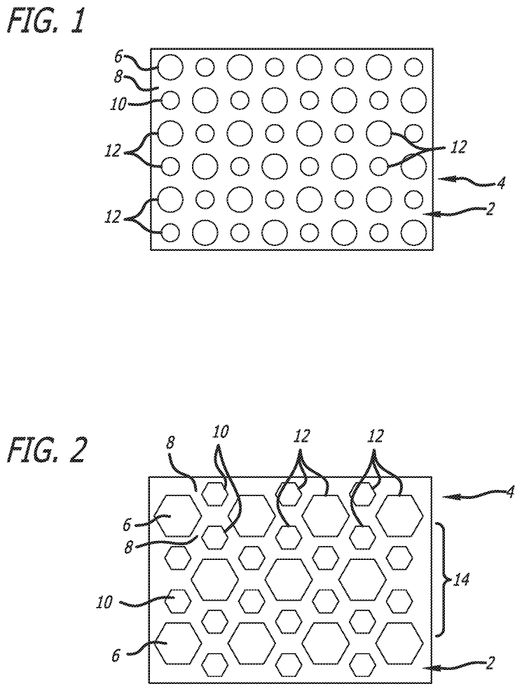

The pouches and tapes of the invention can be porous. As shown in FIGS. 1 and 2, porous pouches can be formed by punching holes or laser cutting holes in the films that form the pouch. Porous pouches can also by forming woven or non-woven fibers made from the biodegradable polymers into pouches. Depending on the fibers and the weave, such pouches may be microporous. As an example, the pouch need not completely encase or surround the IMD. An IMD is thus substantially encapsulated, encased, surrounded or covered when the pouch can hold the device and at least 20%, 30%, 50%, 60%, 75%, 80%, 85%, 90%, 95% or 98% of the device is within the pouch. Porous pouches and partially encased pouches permit contact with tissue and body fluids and are particularly useful with monopole CRM or other IMD devices. Porosity will contribute to the percentage of the IMD covered by the pouch. That is, an IMD is considered to be 50% covered if it is completely surrounded by a pouch that is constructed of a film with 50% voids or holes.

Drugs

Any drug, biological agent, or active ingredient compatible with the process of preparing the pouches and tapes disclosed herein can be incorporated into the pouch or tape, or into one or more of the biodegradable polymer layers that form an embodiment of the invention. Doses of such drugs and agents are known in the art. Those of skill in the art can readily determine the amount of a particular drug to include in described embodiments.

Examples of drugs suitable for use with the present invention include anesthetics, antibiotics (antimicrobials), anti-inflammatory agents, fibrosis-inhibiting agents, anti-scarring agents, leukotriene inhibitors/antagonists, cell growth inhibitors and the like. As used herein, “drugs” is used to include all types of therapeutic agents, whether small molecules or large molecules such as proteins, nucleic acids and the like. The drugs of the invention can be used alone or in combination.

Any pharmaceutically acceptable form of the drugs of the present invention can be employed in the present invention, e.g., the free base or a pharmaceutically acceptable salt or ester thereof. Pharmaceutically acceptable salts, for instance, include sulfate, lactate, acetate, stearate, hydrochloride, tartrate, maleate, citrate, phosphate and the like.

Examples of non-steroidal anti-inflammatories include, but are not limited to, naproxen, ketoprofen, ibuprofen as well as diclofenac; celecoxib; sulindac; diflunisal; piroxicam; indomethacin; etodolac; meloxicam; r-flurbiprofen; mefenamic; nabumetone; tolmetin, and sodium salts of each of the foregoing; ketorolac bromethamine; ketorolac bromethamine tromethamine; choline magnesium trisalicylate; rofecoxib; valdecoxib; lumiracoxib; etoricoxib; aspirin; salicylic acid and its sodium salt; salicylate esters of alpha, beta, gamma-tocopherols and tocotrienols (and all their d, 1, and racemic isomers); and the methyl, ethyl, propyl, isopropyl, n-butyl, sec-butyl, t-butyl, esters of acetylsalicylic acid.

Examples of anesthetics include, but are not limited to, lidocaine, bupivacaine, and mepivacaine. Further examples of analgesics, anesthetics and narcotics include, but are not limited to acetaminophen, clonidine, benzodiazepine, the benzodiazepine antagonist flumazenil, lidocaine, tramadol, carbamazepine, meperidine, zaleplon, trimipramine maleate, buprenorphine, nalbuphine, pentazocain, fentanyl, propoxyphene, hydromorphone, methadone, morphine, levorphanol, and hydrocodone. Local anesthetics have weak antibacterial properties and can play a dual role in the prevention of acute pain and infection.

Examples of antimicrobials include, but are not limited to, triclosan, chlorhexidine, rifampin, minocycline (or other tetracycline derivative), vancomycin, gentamycin, cephalosporins and the like. In preferred embodiments the coatings contain rifampin and another antimicrobial agent, especially a tetracycline derivative. In another preferred embodiment, the coatings contains a cephalosporin and another antimicrobial agent. Preferred combinations include rifampin and minocycline, rifampin and gentamycin, and rifampin and minocycline.

Further antimicrobials include aztreonam; cefotetan and its disodium salt; loracarbef; cefoxitin and its sodium salt; cefazolin and its sodium salt; cefaclor; ceftibuten and its sodium salt; ceftizoxime; ceftizoxime sodium salt; cefoperazone and its sodium salt; cefuroxime and its sodium salt; cefuroxime axetil; cefprozil; ceftazidime; cefotaxime and its sodium salt; cefadroxil; ceftazidime and its sodium salt; cephalexin; cefamandole nafate; cefepime and its hydrochloride, sulfate, and phosphate salt; cefdinir and its sodium salt; ceftriaxone and its sodium salt; cefixime and its sodium salt; cefpodoxime proxetil; meropenem and its sodium salt; imipenem and its sodium salt; cilastatin and its sodium salt; azithromycin; clarithromycin; dirithromycin; erythromycin and hydrochloride, sulfate, or phosphate salts ethylsuccinate, and stearate forms thereof; clindamycin; clindamycin hydrochloride, sulfate, or phosphate salt; lincomycin and hydrochloride, sulfate, or phosphate salt thereof; tobramycin and its hydrochloride, sulfate, or phosphate salt; streptomycin and its hydrochloride, sulfate, or phosphate salt; vancomycin and its hydrochloride, sulfate, or phosphate salt; neomycin and its hydrochloride, sulfate, or phosphate salt; acetyl sulfisoxazole; colistimethate and its sodium salt; quinupristin; dalfopristin; amoxicillin; ampicillin and its sodium salt; clavulanic acid and its sodium or potassium salt; penicillin G; penicillin G benzathine, or procaine salt; penicillin G sodium or potassium salt; carbenicillin and its disodium or indanyl disodium salt; piperacillin and its sodium salt; ticarcillin and its disodium salt; sulbactam and its sodium salt; moxifloxacin; ciprofloxacin; ofloxacin; levofloxacins; norfloxacin; gatifloxacin; trovafloxacin mesylate; alatrofloxacin mesylate; trimethoprim; sulfamethoxazole; demeclocycline and its hydrochloride, sulfate, or phosphate salt; doxycycline and its hydrochloride, sulfate, or phosphate salt; minocycline and its hydrochloride, sulfate, or phosphate salt; tetracycline and its hydrochloride, sulfate, or phosphate salt; oxytetracycline and its hydrochloride, sulfate, or phosphate salt; chlortetracycline and its hydrochloride, sulfate, or phosphate salt; metronidazole; dapsone; atovaquone; rifabutin; linezolide; polymyxin B and its hydrochloride, sulfate, or phosphate salt; sulfacetamide and its sodium salt; and clarithromycin.

Examples of antifungals include amphotericin B; pyrimethamine; flucytosine; caspofungin acetate; fluconazole; griseofulvin; terbinafin and its hydrochloride, sulfate, or phosphate salt; ketoconazole; micronazole; clotrimazole; econazole; ciclopirox; naftifine; and itraconazole.

Other drugs that can be incorporated into the coatings on the mesh pouches disclosed herein include, but are not limited to, keflex, acyclovir, cephradine, malphalen, procaine, ephedrine, adriamycin, daunomycin, plumbagin, atropine, quinine, digoxin, quinidine, biologically active peptides, cephradine, cephalothin, cis-hydroxy-L-proline, melphalan, penicillin V, aspirin, nicotinic acid, chemodeoxycholic acid, chlorambucil, paclitaxel, sirolimus, cyclosporins, 5-flurouracil and the like.

Additional, drugs include those that act as angiogenensis inhibitors or inhibit cell growth such as epidermal growth factor, PDGF, VEGF, FGF (fibroblast growth factor) and the like. These drugs include anti-growth factor antibodies (neutrophilin-1), growth factor receptor-specific inhibitors such as endostatin and thalidomide.

Examples of anti-inflammatory compounds suitable for use in disclosed embodiments include, but are not limited to, anecortive acetate; tetrahydrocortisol, 4,9(11)-pregnadien-17.alpha.,21-diol-3,20-dione and its -21-acetate salt; 11-epicortisol; 17.alpha.-hydroxyprogesterone; tetrahydrocortexolone; cortisona; cortisone acetate; hydrocortisone; hydrocortisone acetate; fludrocortisone; fludrocortisone acetate; fludrocortisone phosphate; prednisone; prednisolone; prednisolone sodium phosphate; methylprednisolone; methylprednisolone acetate; methylprednisolone, sodium succinate; triamcinolone; triamcinolone-16,21-diacetate; triamcinolone acetonide and its -21-acetate, -21-disodium phosphate, and -21-hemisuccinate forms; triamcinolone benetonide; triamcinolone hexacetonide; fluocinolone and fluocinolone acetate; dexamethasone and its -21-acetate, -21-(3,3-dimethylbutyrate), -21-phosphate disodium salt, -21-diethylaminoacetate, -21-isonicotinate, -21-dipropionate, and -21-palmitate forms; betamethasone and its -21-acetate, -21-adamantoate, -17-benzoate, -17,21-dipropionate, -17-valerate, and -21-phosphate disodium salts; beclomethasone; beclomethasone dipropionate; diflorasone; diflorasone diacetate; mometasone furoate; and acetazolamide.

Examples of leukotriene inhibitors/antagonists include, but are not limited to, leukotriene receptor antagonists such as acitazanolast, iralukast, montelukast, pranlukast, verlukast, zafirlukast, and zileuton.

Another useful drug that can be incorporated into the coatings of the invention is sodium 2-mercaptoethane sulfonate (Mesna). Mesna has been shown to diminish myofibroblast formation in animal studies of capsular contracture with breast implants [Ajmal et al. (2003) Plast. Reconstr. Surg. 112:1455-1461] and may thus act as an anti-fibrosis agent.

CRMs and Other IMDs

The CRMs and other IMDs used with the disclosed embodiments can include but are not limited to pacemakers, defibrillators, implantable access systems, neurostimulators, other stimulation devices, ventricular assist devices, infusion pumps or other implantable devices (or implantable components thereof) for delivering medication, hydrating solutions or other fluids, intrathecal delivery systems, pain pumps, or any other implantable system to provide drugs or electrical stimulation to a body part.

Disclosed pouches and tapes can thus be designed to fit a wide range of pacemakers and implantable defibrillators from a variety of manufacturers. Sizes of the CRMs vary and typically size ranges are listed in the following Table:

CRM Devices

| | | | | Size |
|---|---|---|---|---|
| Manufacturer | Device | Type | Model | (H″ × L″ × W″) |
| Medtronic | EnPulse Pacing | Pacing system | E2DR01 | 1.75 × 2 × 0.33 |
| | system | | | |
| Medtronic | EnPulse Pacing | Pacing system | E2DR21 | 1.75 × 1.63 × 0.33 |
| | system | | | |
| Medtronic | EnRhythm | Pacing system | P1501DR | 1.77 × 2 × 0.31 |
| | Pacing system | | | |
| Medtronic | AT500 Pacing | Pacing system | AT501 | 1.75 × 2.38 × 0.33 |
| | system | | | |
| Medtronic | Kappa DR900 | Pacing system | DR900, DR700 | 1.75-2 × 1.75-2 × 0.33 |
| | & 700 series | | | |
| Medtronic | Kappa DR900 | Pacing system | SR900, SR700 | 1.5-175 × 1.75-2 × 0.33 |
| | & 700 series | | | |
| Medtronic | Sigma | Pacing system | D300, D200, D303, | 1.75 × 2 × 0.33 |
| | | | D203 | |
| Medtronic | Sigma | Pacing system | DR300, DR200, | 1.75-2 × 2 × 0.33 |
| | | | DR303, DR306, | |
| | | | DR203 | |
| Medtronic | Sigma | Pacing system | VDD300, VDD303 | 1.75 × 1.75 × 0.33 |
| Medtronic | Sigma | Pacing system | S300, S200, S100, | 1.63 × 2 × 0.33 |
| | | | S303, S203, S103, | |
| | | | S106, VVI-103 | |
| Medtronic | Sigma SR | Pacing system | SR300, S200, | 1.63 × 2 × 0.33 |
| | | | SR303, SR306, | |
| | | | SR203 | |
| Medtronic | Entrust | Defibrillator | D154VRC 35J | 2.44 × 2 × 0.6 |
| Medtronic | Maximo & | Defibrillator | | Size of a pager |
| | Marquis family | | | |
| Medtronic | Gem family | Defibrillator | III T, III R, III R, II | Size of a pager |
| | | | R, II VR | |
| Guidant | Contak Renewal | Pacing system | H120, H125 | 2.13 × 1.77 × 0.33 |
| | TR | | | |
| St. Jude | Identity | Pacing system | ADx DR, ADx SR, | 1.6-1.73 × 1.73-2.05 × 0.24 |
| | | | ADx XI, ADx VDR | |
| St. Jude | Integrity | Pacing system | ADx DR, ADx SR | 1.6-1.73 × 1.73-2.05 × 0.24 |

Implantable neurostimulators are similar to pacemakers in that the devices generate electrical impulses. These devices send electrical signals via leads to the spine and brain to treat pain and other neurological disorders. For example, when the leads are implanted in the spine, the neurostimulation can be used to treat chronic pain (especially back and spinal pain); when the leads are implanted in the brain, the neurostimulation can be used to treat epilepsy and essential tremor including the tremors associated with Parkinson's disease and other neurological disorders. Neurostimulation can be used to treat severe, chronic nausea and vomiting as well as urological disorders. For the former, electrical impulses are sent to the stomach; for the latter, the electrical impulses are sent to the sacral nerves in the lower back. The implant location of the neurostimulator varies by application but, in all cases, is placed under the skin and is susceptible to infection at the time of implantation and post-implantation. Likewise, reintervention and replacement of batteries in the neurostimulators can occur at regular intervals.

Disclosed pouches of the invention can thus be designed to fit a wide range of neurostimulators from a variety of manufacturers (see the following Table). Sizes of the neurostimulators vary and typically size ranges are listed in the following Table:

Neurostimulators

| Manufacturer | Device | Type | Model | Size (H″ × L″ × W″) |
|---|---|---|---|---|
| Medtronic | InterStim | Neurostimulation | 3023 | 2.17 × 2.4 × 0.39 |
| | INS | | | |
| Medtronic | InterStim | Neurostimulation | 3058 | 1.7 × 2.0 × 0.3 |
| | INS II | | | |
| Medtronic | RESTORE | Neurostimulation | 37711 | 2.56 × 1.93 × 0.6 |
| Advanced | Precision | Neurostimulation/Spinal | | 2.09 × 1.70 × 0.35 |
| Bionics | IPG | Cord Stimulator | | |
| (Boston | | | | |
| Scientific) | | | | |
| Cyberonics | VNS | Neurostimulation/Epilepsy | 102 | 2.03 × 2.06 × 0.27 |
| | Therapy | | | |
| | system | | | |
| Cyberonics | VNS | Neurostimulation/Epilepsy | 102R | 2.03 × 2.32 × 0.27 |
| | Therapy | | | |
| | system | | | |
| ANS (St. Jude) | Eon | Neurostimulation | | Comparable to |
| | | | | Medtronic Restore |
| ANS (St. Jude) | Genesis RC | Neurostimulation | | Comparable to |
| | | | | Medtronic Restore |
| ANS (St. Jude) | Genesis XP | Neurostimulation | | Comparable to |
| | | | | Medtronic Restore |

The fully resorbable polymer pouches of the invention can be prepared using any of the foregoing biodegradable polymers, and preferably using any one or more of the tyrosine-based polyarylates described above. Such polymers are dissolved in appropriate solvents to cast films, prepare spray coating solutions, electrospinning solutions, molding solutions and the like for forming a fully resorbable polymer film.

Fully resorbable pouches, as films, meshes, non-wovens and the like, can be created by several means: by spray coating a substrate, thermal or solvent casting, weaving, knitting or electrospinning, dip coating, extrusion, or molding. Sheets can be formed into a pouch configuration by thermoforming, or mechanical forming and heat setting, or adhesive, thermal or ultrasonic assembly. Pouches can be constructed directly from resorbable polymer by dip or spray coating a pre-formed shape, or injection or compression molding into the finished shape.

The biodegradable polymers can be formed into multi-layered fully resorbable polymer films, each layer containing the same or different polymers, the same or different drugs, and the same or different amounts of polymers or drugs. For example, a first film layer can contain drug, while the film layer coating layer contains either no drug or a lower concentration of drug. For example, a multilayer film can be made by casting a first layer, allowing it to dry, and casting a second or successive layer onto the first layer, allowing each layer to dry before casting the next layer.

The pouches can be shaped to fit relatively snugly or more loosely around an IMD. For example, in an embodiment the IMD is capable of being folded, and each half interlocks with the other half to secure the shell around the device and hold the device within the clamshell. Additionally, the clamshell has a space or opening sufficient to allow the leads from the device to pass through the clamshell. The number of spaces or opening in the pouch that are provided can match the number and placement of the leads or other tubes extending from the CRM or other IMD, as applicable for the relevant device. The pouches are constructed with polymers from the group described herein that are selected to have elastomeric properties if is desirable to have a pouch that fits tightly over the IMD.

The films can be laser cut to produce the desired shaped and sized pouches, coverings and the like. Two pieces can be sealed, by heat, by ultrasound or other method known in the art, leaving one side open to permit insertion of the device at the time of the surgical procedure.

In embodiments, the substrate can comprise electrodes or microcells. Each electrode or microcell can be or comprise a conductive metal. In embodiments, the electrodes or microcells can comprise any electrically-conductive material, for example, an electrically conductive hydrogel, metals, electrolytes, superconductors, semiconductors, plasmas, and nonmetallic conductors such as graphite and conductive polymers. Electrically conductive metals can comprise silver, copper, gold, aluminum, molybdenum, zinc, lithium, tungsten, brass, carbon, nickel, iron, palladium, platinum, tin, bronze, carbon steel, lead, titanium, stainless steel, mercury, Fe/Cr alloys, and the like. The electrodes can be solid, coated or plated with a different metal such as aluminum, gold, platinum or silver.

In certain embodiments, reservoir or electrode geometry can comprise circles, polygons, lines, zigzags, ovals, stars, or any suitable variety of shapes. This provides the ability to design/customize surface electric field shapes as well as depth of penetration. For example. In embodiments it can be desirable to employ an electric field of greater strength or depth in an area to achieve optimal treatment. In another embodiment, the desirable strength of an electric field be employed within a three dimensional material such as a hydrogel or solid object.

Reservoir or electrode or dot sizes and concentrations can vary, as these variations can allow for changes in the properties of the electric field created by embodiments of the invention. Certain embodiments provide an electric field at about 1 Volt and then, under normal tissue loads with resistance of 100 k to 300K ohms, produce a current in the range of 10 microamperes.

In other embodiments, a system can be provided which comprises an external battery or power source. For example, an AC power source can be of any wave form, such as a sine wave, a triangular wave, or a square wave. AC power can be of any frequency such as for example 50 Hz or 60 Hz, or the like. AC power can also be of any voltage, such as for example 120 volts, 220 volts, or the like. In embodiments an AC power source can be electronically modified, such as for example having the voltage reduced, prior to use. Embodiments can comprise an on/off switch. Embodiments can comprise an indicator, for example a visual indicator, for example an LED, to confirm that the device is functioning correctly.

In various embodiments the difference of the standard potentials of the electrodes or dots or reservoirs can be in a range from about 0.05 V to approximately about 5.0 V. For example, the standard potential can be about 0.05 V, about 0.06 V, about 0.07 V, about 0.08 V, about 0.09 V, about 0.1 V, about 0.2 V, about 0.3 V, about 0.4 V, about 0.5 V, about 0.6 V, about 0.7 V, about 0.8 V, about 0.9 V, about 1.0 V, about 1.1 V, about 1.2 V, about 1.3 V, about 1.4 V, about 1.5 V, about 1.6 V, about 1.7 V, about 1.8 V, about 1.9 V, about 2.0 V, about 2.1 V, about 2.2 V, about 2.3 V, about 2.4 V, about 2.5 V, about 2.6 V, about 2.7 V, about 2.8 V, about 2.9 V, about 3.0 V, about 3.1 V, about 3.2 V, about 3.3 V, about 3.4 V, about 3.5 V, about 3.6 V, about 3.7 V, about 3.8 V, about 3.9 V, about 4.0 V, about 4.1 V, about 4.2 V, about 4.3 V, about 4.4 V, about 4.5 V, about 4.6 V, about 4.7 V, about 4.8 V, about 4.9 V, about 5.0 V, about 5.1 V, about 5.2 V, about 5.3 V, about 5.4 V, about 5.5 V, about 5.6 V, about 5.7 V, about 5.8 V, about 5.9 V, about 6.0 V, about 6.1 V, about 6.2 V, about 6.3 V, about 6.4 V, about 6.5 V, about 6.6 V, about 6.7 V, about 6.8 V, about 6.9 V, about 7.0 V, about 7.1 V, about 7.2 V, about 7.3 V, about 7.4 V, about 7.5 V, about 7.6 V, about 7.7 V, about 7.8 V, about 7.9 V, about 8.0 V, about 8.1 V, about 8.2 V, about 8.3 V, about 8.4 V, about 8.5 V, about 8.6 V, about 8.7 V, about 8.8 V, about 8.9 V, about 9.0 V, or the like.

In embodiments, systems and devices disclosed herein can produce a low level electric current of between for example about 1 and about 200 micro-amperes, between about 10 and about 190 micro-amperes, between about 20 and about 180 micro-amperes, between about 30 and about 170 micro-amperes, between about 40 and about 160 micro-amperes, between about 50 and about 150 micro-amperes, between about 60 and about 140 micro-amperes, between about 70 and about 130 micro-amperes, between about 80 and about 120 micro-amperes, between about 90 and about 100 micro-amperes, between about 100 and about 150 micro-amperes, between about 150 and about 200 micro-amperes, between about 200 and about 250 micro-amperes, between about 250 and about 300 micro-amperes, between about 300 and about 350 micro-amperes, between about 350 and about 400 micro-amperes, between about 400 and about 450 micro-amperes, between about 450 and about 500 micro-amperes, between about 500 and about 550 micro-amperes, between about 550 and about 600 micro-amperes, between about 600 and about 650 micro-amperes, between about 650 and about 700 micro-amperes, between about 700 and about 750 micro-amperes, between about 750 and about 800 micro-amperes, between about 800 and about 850 micro-amperes, between about 850 and about 900 micro-amperes, between about 900 and about 950 micro-amperes, between about 950 and about 1000 micro-amperes (1 milli-amp [mA]), between about 1.0 and about 1.1 mA, between about 1.1 and about 1.2 mA, between about 1.2 and about 1.3 mA, between about 1.3 and about 1.4 mA, between about 1.4 and about 1.5 mA, between about 1.5 and about 1.6 mA, between about 1.6 and about 1.7 mA, between about 1.7 and about 1.8 mA, between about 1.8 and about 1.9 mA, between about 1.9 and about 2.0 mA, between about 2.0 and about 2.1 mA, between about 2.1 and about 2.2 mA, between about 2.2 and about 2.3 mA, between about 2.3 and about 2.4 mA, between about 2.4 and about 2.5 mA, between about 2.5 and about 2.6 mA, between about 2.6 and about 2.7 mA, between about 2.7 and about 2.8 mA, between about 2.8 and about 2.9 mA, between about 2.9 and about 3.0 mA, between about 3.0 and about 3.1 mA, between about 3.1 and about 3.2 mA, between about 3.2 and about 3.3 mA, between about 3.3 and about 3.4 mA, between about 3.4 and about 3.5 mA, between about 3.5 and about 3.6 mA, between about 3.6 and about 3.7 mA, between about 3.7 and about 3.8 mA, between about 3.8 and about 3.9 mA, between about 3.9 and about 4.0 mA, between about 4.0 and about 4.1 mA, between about 4.1 and about 4.2 mA, between about 4.2 and about 4.3 mA, between about 4.3 and about 4.4 mA, between about 4.4 and about 4.5 mA, between about 4.5 and about 5.0 mA, between about 5.0 and about 5.5 mA, between about 5.5 and about 6.0 mA, between about 6.0 and about 6.5 mA, between about 6.5 and about 7.0 mA, between about 7.5 and about 8.0 mA, between about 8.0 and about 8.5 mA, between about 8.5 and about 9.0 mA, between about 9.0 and about 9.5 mA, between about 9.5 and about 10.0 mA, between about 10.0 and about 10.5 mA, between about 10.5 and about 11.0 mA, between about 11.0 and about 11.5 mA, between about 11.5 and about 12.0 mA, between about 12.0 and about 12.5 mA, between about 12.5 and about 13.0 mA, between about 13.0 and about 13.5 mA, between about 13.5 and about 14.0 mA, between about 14.0 and about 14.5 mA, between about 14.5 and about 15.0 mA, or the like.

In embodiments, systems and devices disclosed herein can produce a low level electric current of between for example about 1 and about 400 micro-amperes, between about 20 and about 380 micro-amperes, between about 40 and about 360 micro-amperes, between about 60 and about 340 micro-amperes, between about 80 and about 320 micro-amperes, between about 100 and about 300 micro-amperes, between about 120 and about 280 micro-amperes, between about 140 and about 260 micro-amperes, between about 160 and about 240 micro-amperes, between about 180 and about 220 micro-amperes, or the like.

In embodiments, systems and devices disclosed herein can produce a low level electric current of between for example about 1 micro-ampere and about 1 milli-ampere, between about 50 and about 800 micro-amperes, between about 200 and about 600 micro-amperes, between about 400 and about 500 micro-amperes, or the like.

In embodiments, systems and devices disclosed herein can produce a low level electric current of about 10 micro-amperes, about 20 micro-amperes, about 30 micro-amperes, about 40 micro-amperes, about 50 micro-amperes, about 60 micro-amperes, about 70 micro-amperes, about 80 micro-amperes, about 90 micro-amperes, about 100 micro-amperes, about 110 micro-amperes, about 120 micro-amperes, about 130 micro-amperes, about 140 micro-amperes, about 150 micro-amperes, about 160 micro-amperes, about 170 micro-amperes, about 180 micro-amperes, about 190 micro-amperes, about 200 micro-amperes, about 210 micro-amperes, about 220 micro-amperes, about 240 micro-amperes, about 260 micro-amperes, about 280 micro-amperes, about 300 micro-amperes, about 320 micro-amperes, about 340 micro-amperes, about 360 micro-amperes, about 380 micro-amperes, about 400 micro-amperes, about 450 micro-amperes, about 500 micro-amperes, about 550 micro-amperes, about 600 micro-amperes, about 650 micro-amperes, about 700 micro-amperes, about 750 micro-amperes, about 800 micro-amperes, about 850 micro-amperes, about 900 micro-amperes, about 950 micro-amperes, about 1 milli-ampere (mA), about 1.1 mA, about 1.2 mA, about 1.3 mA, about 1.4 mA, about 1.5 mA, about 1.6 mA, about 1.7 mA, about 1.8 mA, about 1.9 mA, about 2.0 mA, about 2.1 mA, about 2.2 mA, about 2.3 mA, about 2.4 mA, about 2.5 mA, about 2.6 mA, about 2.7 mA, about 2.8 mA, about 2.9 mA, about 3.0 mA, about 3.1 mA, about 3.2 mA, about 3.3 mA, about 3.4 mA, about 3.5 mA, about 3.6 mA, about 3.7 mA, about 3.8 mA, about 3.9 mA, about 4.0 mA, about 4.1 mA, about 4.2 mA, about 4.3 mA, about 4.4 mA, about 4.5 mA, about 4.6 mA, about 4.7 mA, about 4.8 mA, about 4.9 mA, about 5.0 mA, about 5.1 mA, about 5.2 mA, about 5.3 mA, about 5.4 mA, about 5.5 mA, about 5.6 mA, about 5.7 mA, about 5.8 mA, about 5.9 mA, about 6.0 mA, about 6.1 mA, about 4.2 mA, about 6.3 mA, about 6.4 mA, about 6.5 mA, about 6.6 mA, about 6.7 mA, about 6.8 mA, about 6.9 mA, about 7.0 mA, about 7.1 mA, about 7.2 mA, about 7.3 mA, about 7.4 mA, about 7.5 mA, about 7.6 mA, about 7.7 mA, about 7.8 mA, about 7.9 mA, about 8.0 mA, about 8.1 mA, about 8.2 mA, about 8.3 mA, about 8.4 mA, about 8.5 mA, about 8.6 mA, about 8.7 mA, about 8.8 mA, about 8.9 mA, about 9.0 mA, about 9.1 mA, about 9.2 mA, about 9.3 mA, about 9.4 mA, about 9.5 mA, about 9.6 mA, about 9.7 mA, about 9.8 mA, about 9.9 mA, about 10.0 mA, about 10.1 mA, about 10.2 mA, about 10.3 mA, about 10.4 mA, about 10.5 mA, about 10.6 mA, about 10.7 mA, about 10.8 mA, about 10.9 mA, about 11.0 mA, about 11.1 mA, about 11.2 mA, about 11.3 mA, about 11.4 mA, about 11.5 mA, about 11.6 mA, about 11.7 mA, about 11.8 mA, about 11.9 mA, about 12.0 mA, about 12.1 mA, about 12.2 mA, about 12.3 mA, about 12.4 mA, about 12.5 mA, about 12.6 mA, about 12.7 mA, about 12.8 mA, about 12.9 mA, about 13.0 mA, about 13.1 mA, about 13.2 mA, about 13.3 mA, about 13.4 mA, about 13.5 mA, about 13.6 mA, about 13.7 mA, about 13.8 mA, about 13.9 mA, about 14.0 mA, about 14.1 mA, about 14.2 mA, about 14.3 mA, about 14.4 mA, about 14.5 mA, about 14.6 mA, about 14.7 mA, about 14.8 mA, about 14.9 mA, about 15.0 mA, about 15.1 mA, about 15.2 mA, about 15.3 mA, about 15.4 mA, about 15.5 mA, about 15.6 mA, about 15.7 mA, about 15.8 mA, or the like.

In embodiments, the disclosed systems and devices can produce a low level electric current of not more than 10 micro-amperes, or not more than about 20 micro-amperes, not more than about 30 micro-amperes, not more than about 40 micro-amperes, not more than about 50 micro-amperes, not more than about 60 micro-amperes, not more than about 70 micro-amperes, not more than about 80 micro-amperes, not more than about 90 micro-amperes, not more than about 100 micro-amperes, not more than about 110 micro-amperes, not more than about 120 micro-amperes, not more than about 130 micro-amperes, not more than about 140 micro-amperes, not more than about 150 micro-amperes, not more than about 160 micro-amperes, not more than about 170 micro-amperes, not more than about 180 micro-amperes, not more than about 190 micro-amperes, not more than about 200 micro-amperes, not more than about 210 micro-amperes, not more than about 220 micro-amperes, not more than about 230 micro-amperes, not more than about 240 micro-amperes, not more than about 250 micro-amperes, not more than about 260 micro-amperes, not more than about 270 micro-amperes, not more than about 280 micro-amperes, not more than about 290 micro-amperes, not more than about 300 micro-amperes, not more than about 310 micro-amperes, not more than about 320 micro-amperes, not more than about 340 micro-amperes, not more than about 360 micro-amperes, not more than about 380 micro-amperes, not more than about 400 micro-amperes, not more than about 420 micro-amperes, not more than about 440 micro-amperes, not more than about 460 micro-amperes, not more than about 480 micro-amperes, not more than about 500 micro-amperes, not more than about 520 micro-amperes, not more than about 540 micro-amperes, not more than about 560 micro-amperes, not more than about 580 micro-amperes, not more than about 600 micro-amperes, not more than about 620 micro-amperes, not more than about 640 micro-amperes, not more than about 660 micro-amperes, not more than about 680 micro-amperes, not more than about 700 micro-amperes, not more than about 720 micro-amperes, not more than about 740 micro-amperes, not more than about 760 micro-amperes, not more than about 780 micro-amperes, not more than about 800 micro-amperes, not more than about 820 micro-amperes, not more than about 840 micro-amperes, not more than about 860 micro-amperes, not more than about 880 micro-amperes, not more than about 900 micro-amperes, not more than about 920 micro-amperes, not more than about 940 micro-amperes, not more than about 960 micro-amperes, not more than about 980 micro-amperes, not more than about 1 milli-ampere (mA), not more than about 1.1 mA, not more than about 1.2 mA, not more than about 1.3 mA, not more than about 1.4 mA, not more than about 1.5 mA, not more than about 1.6 mA, not more than about 1.7 mA, not more than about 1.8 mA, not more than about 1.9 mA, not more than about 2.0 mA, not more than about 2.1 mA, not more than about 2.2 mA, not more than about 2.3 mA, not more than about 2.4 mA, not more than about 2.5 mA, not more than about 2.6 mA, not more than about 2.7 mA, not more than about 2.8 mA, not more than about 2.9 mA, not more than about 3.0 mA, not more than about 3.1 mA, not more than about 3.2 mA, not more than about 3.3 mA, not more than about 3.4 mA, not more than about 3.5 mA, not more than about 3.6 mA, not more than about 3.7 mA, not more than about 3.8 mA, not more than about 3.9 mA, not more than about 4.0 mA, not more than about 4.1 mA, not more than about 4.2 mA, not more than about 4.3 mA, not more than about 4.4 mA, not more than about 4.5 mA, not more than about 4.6 mA, not more than about 4.7 mA, not more than about 4.8 mA, not more than about 4.9 mA, not more than about 5.0 mA, not more than about 5.1 mA, not more than about 5.2 mA, not more than about 5.3 mA, not more than about 5.4 mA, not more than about 5.5 mA, not more than about 5.6 mA, not more than about 5.7 mA, not more than about 5.8 mA, not more than about 5.9 mA, not more than about 6.0 mA, not more than about 6.1 mA, not more than about 4.2 mA, not more than about 6.3 mA, not more than about 6.4 mA, not more than about 6.5 mA, not more than about 6.6 mA, not more than about 6.7 mA, not more than about 6.8 mA, not more than about 6.9 mA, not more than about 7.0 mA, not more than about 7.1 mA, not more than about 7.2 mA, not more than about 7.3 mA, not more than about 7.4 mA, not more than about 7.5 mA, not more than about 7.6 mA, not more than about 7.7 mA, not more than about 7.8 mA, not more than about 7.9 mA, not more than about 8.0 mA, not more than about 8.1 mA, not more than about 8.2 mA, not more than about 8.3 mA, not more than about 8.4 mA, not more than about 8.5 mA, not more than about 8.6 mA, not more than about 8.7 mA, not more than about 8.8 mA, not more than about 8.9 mA, not more than about 9.0 mA, not more than about 9.1 mA, not more than about 9.2 mA, not more than about 9.3 mA, not more than about 9.4 mA, not more than about 9.5 mA, not more than about 9.6 mA, not more than about 9.7 mA, not more than about 9.8 mA, not more than about 9.9 mA, not more than about 10.0 mA, not more than about 10.1 mA, not more than about 10.2 mA, not more than about 10.3 mA, not more than about 10.4 mA, not more than about 10.5 mA, not more than about 10.6 mA, not more than about 10.7 mA, not more than about 10.8 mA, not more than about 10.9 mA, not more than about 11.0 mA, not more than about 11.1 mA, not more than about 11.2 mA, not more than about 11.3 mA, not more than about 11.4 mA, not more than about 11.5 mA, not more than about 11.6 mA, not more than about 11.7 mA, not more than about 11.8 mA, not more than about 11.9 mA, not more than about 12.0 mA, not more than about 12.1 mA, not more than about 12.2 mA, not more than about 12.3 mA, not more than about 12.4 mA, not more than about 12.5 mA, not more than about 12.6 mA, not more than about 12.7 mA, not more than about 12.8 mA, not more than about 12.9 mA, not more than about 13.0 mA, not more than about 13.1 mA, not more than about 13.2 mA, not more than about 13.3 mA, not more than about 13.4 mA, not more than about 13.5 mA, not more than about 13.6 mA, not more than about 13.7 mA, not more than about 13.8 mA, not more than about 13.9 mA, not more than about 14.0 mA, not more than about 14.1 mA, not more than about 14.2 mA, not more than about 14.3 mA, not more than about 14.4 mA, not more than about 14.5 mA, not more than about 14.6 mA, not more than about 14.7 mA, not more than about 14.8 mA, not more than about 14.9 mA, not more than about 15.0 mA, not more than about 15.1 mA, not more than about 15.2 mA, not more than about 15.3 mA, not more than about 15.4 mA, not more than about 15.5 mA, not more than about 15.6 mA, not more than about 15.7 mA, not more than about 15.8 mA, and the like.

In embodiments, systems and devices disclosed herein can produce a low level electric current of not less than 10 micro-amperes, not less than 20 micro-amperes, not less than 30 micro-amperes, not less than 40 micro-amperes, not less than 50 micro-amperes, not less than 60 micro-amperes, not less than 70 micro-amperes, not less than 80 micro-amperes, not less than 90 micro-amperes, not less than 100 micro-amperes, not less than 110 micro-amperes, not less than 120 micro-amperes, not less than 130 micro-amperes, not less than 140 micro-amperes, not less than 150 micro-amperes, not less than 160 micro-amperes, not less than 170 micro-amperes, not less than 180 micro-amperes, not less than 190 micro-amperes, not less than 200 micro-amperes, not less than 210 micro-amperes, not less than 220 micro-amperes, not less than 230 micro-amperes, not less than 240 micro-amperes, not less than 250 micro-amperes, not less than 260 micro-amperes, not less than 270 micro-amperes, not less than 280 micro-amperes, not less than 290 micro-amperes, not less than 300 micro-amperes, not less than 310 micro-amperes, not less than 320 micro-amperes, not less than 330 micro-amperes, not less than 340 micro-amperes, not less than 350 micro-amperes, not less than 360 micro-amperes, not less than 370 micro-amperes, not less than 380 micro-amperes, not less than 390 micro-amperes, not less than 400 micro-amperes, not less than about 420 micro-amperes, not less than about 440 micro-amperes, not less than about 460 micro-amperes, not less than about 480 micro-amperes, not less than about 500 micro-amperes, not less than about 520 micro-amperes, not less than about 540 micro-amperes, not less than about 560 micro-amperes, not less than about 580 micro-amperes, not less than about 600 micro-amperes, not less than about 620 micro-amperes, not less than about 640 micro-amperes, not less than about 660 micro-amperes, not less than about 680 micro-amperes, not less than about 700 micro-amperes, not less than about 720 micro-amperes, not less than about 740 micro-amperes, not less than about 760 micro-amperes, not less than about 780 micro-amperes, not less than about 800 micro-amperes, not less than about 820 micro-amperes, not less than about 840 micro-amperes, not less than about 860 micro-amperes, not less than about 880 micro-amperes, not less than about 900 micro-amperes, not less than about 920 micro-amperes, not less than about 940 micro-amperes, not less than about 960 micro-amperes, not less than about 980 micro-amperes, not less than about 1 milli-ampere (mA), not less than about 1.1 mA, not less than about 1.2 mA, not less than about 1.3 mA, not less than about 1.4 mA, not less than about 1.5 mA, not less than about 1.6 mA, not less than about 1.7 mA, not less than about 1.8 mA, not less than about 1.9 mA, not less than about 2.0 mA, not less than about 2.1 mA, not less than about 2.2 mA, not less than about 2.3 mA, not less than about 2.4 mA, not less than about 2.5 mA, not less than about 2.6 mA, not less than about 2.7 mA, not less than about 2.8 mA, not less than about 2.9 mA, not less than about 3.0 mA, not less than about 3.1 mA, not less than about 3.2 mA, not less than about 3.3 mA, not less than about 3.4 mA, not less than about 3.5 mA, not less than about 3.6 mA, not less than about 3.7 mA, not less than about 3.8 mA, not less than about 3.9 mA, not less than about 4.0 mA, not less than about 4.1 mA, not less than about 4.2 mA, not less than about 4.3 mA, not less than about 4.4 mA, not less than about 4.5 mA, not less than about 4.6 mA, not less than about 4.7 mA, not less than about 4.8 mA, not less than about 4.9 mA, not less than about 5.0 mA, not less than about 5.1 mA, not less than about 5.2 mA, not less than about 5.3 mA, not less than about 5.4 mA, not less than about 5.5 mA, not less than about 5.6 mA, not less than about 5.7 mA, not less than about 5.8 mA, not less than about 5.9 mA, not less than about 6.0 mA, not less than about 6.1 mA, not less than about 4.2 mA, not less than about 6.3 mA, not less than about 6.4 mA, not less than about 6.5 mA, not less than about 6.6 mA, not less than about 6.7 mA, not less than about 6.8 mA, not less than about 6.9 mA, not less than about 7.0 mA, not less than about 7.1 mA, not less than about 7.2 mA, not less than about 7.3 mA, not less than about 7.4 mA, not less than about 7.5 mA, not less than about 7.6 mA, not less than about 7.7 mA, not less than about 7.8 mA, not less than about 7.9 mA, not less than about 8.0 mA, not less than about 8.1 mA, not less than about 8.2 mA, not less than about 8.3 mA, not less than about 8.4 mA, not less than about 8.5 mA, not less than about 8.6 mA, not less than about 8.7 mA, not less than about 8.8 mA, not less than about 8.9 mA, not less than about 9.0 mA, not less than about 9.1 mA, not less than about 9.2 mA, not less than about 9.3 mA, not less than about 9.4 mA, not less than about 9.5 mA, not less than about 9.6 mA, not less than about 9.7 mA, not less than about 9.8 mA, not less than about 9.9 mA, not less than about 10.0 mA, not less than about 10.1 mA, not less than about 10.2 mA, not less than about 10.3 mA, not less than about 10.4 mA, not less than about 10.5 mA, not less than about 10.6 mA, not less than about 10.7 mA, not less than about 10.8 mA, not less than about 10.9 mA, not less than about 11.0 mA, not less than about 11.1 mA, not less than about 11.2 mA, not less than about 11.3 mA, not less than about 11.4 mA, not less than about 11.5 mA, not less than about 11.6 mA, not less than about 11.7 mA, not less than about 11.8 mA, not less than about 11.9 mA, not less than about 12.0 mA, not less than about 12.1 mA, not less than about 12.2 mA, not less than about 12.3 mA, not less than about 12.4 mA, not less than about 12.5 mA, not less than about 12.6 mA, not less than about 12.7 mA, not less than about 12.8 mA, not less than about 12.9 mA, not less than about 13.0 mA, not less than about 13.1 mA, not less than about 13.2 mA, not less than about 13.3 mA, not less than about 13.4 mA, not less than about 13.5 mA, not less than about 13.6 mA, not less than about 13.7 mA, not less than about 13.8 mA, not less than about 13.9 mA, not less than about 14.0 mA, not less than about 14.1 mA, not less than about 14.2 mA, not less than about 14.3 mA, not less than about 14.4 mA, not less than about 14.5 mA, not less than about 14.6 mA, not less than about 14.7 mA, not less than about 14.8 mA, not less than about 14.9 mA, not less than about 15.0 mA, not less than about 15.1 mA, not less than about 15.2 mA, not less than about 15.3 mA, not less than about 15.4 mA, not less than about 15.5 mA, not less than about 15.6 mA, not less than about 15.7 mA, not less than about 15.8 mA, and the like.

In embodiments, disclosed devices can provide an electric field of greater than physiological strength to a depth (as measured from the surface of the device) of, at least 1 mm, 2 mm, 3 mm, 4 mm, 5 mm, 6 mm, 7 mm, 8 mm, 9 mm, 10 mm, 11 mm, 12 mm, 13 mm, 14 mm, 15 mm, 16 mm, 17 mm, 18 mm, 19 mm, 20 mm, 21 mm, 22 mm, 23 mm, 24 mm, 25 mm, 26 mm, 27 mm, 28 mm, 29 mm, 30 mm, 31 mm, 32 mm, 33 mm, 34 mm, 35 mm, 36 mm, 37 mm, 38 mm, 39 mm, 40 mm, or more.

In embodiments the electric field can be extended, for example through the use of a hydrogel. A hydrogel is a network of polymer chains that are hydrophilic. Hydrogels are highly absorbent natural or synthetic polymeric networks. Hydrogels can be configured to contain a high percentage of water (e.g. they can contain over 90% water). Salts can be added to hydrogels to increase the conductivity. Hydrogels can possess a degree of flexibility very similar to natural tissue, due to their significant water content. A hydrogel can be configured in a variety of viscosities. Viscosity is a measurement of a fluid or material's resistance to gradual deformation by shear stress or tensile stress. In embodiments the electrical field can be extended through a semi-liquid hydrogel with a low viscosity such an ointment or a cellular culture medium. In other embodiments the electrical field can be extended through a solid hydrogel with a high viscosity such as a Petri dish, clothing, or material used to manufacture a prosthetic. In general, the hydrogel described herein may be configured to a viscosity of between about 0.5 Pa·s and greater than about 10¹² Pa·s. In embodiments the viscosity of a hydrogel can be, for example, between 0.5 and 10¹² Pa·s, between 1 Pa·s and 10⁶ Pa·s, between 5 and 103 Pa·s, between 10 and 100 Pa·s, between 15 and 90 Pa·s, between 20 and 80 Pa·s, between 25 and 70 Pa·s, between 30 and 60 Pa·s, or the like. In another embodiment, the reservoirs or dots are configured to be same specific gravity as the hydrophilic polymer base of a hydrogel. This embodiment allows the reservoirs or dots to be suspended in the hydrogel for a desired use without the reservoirs or dots being pulled to the bottom of the hydrogels due to other factors such as gravity. In particular, the reservoirs or dots will not settle and the hydrogel can be manufactured and stored for extended periods of times without altering the hydrogel's intended performance.

Embodiments can include coatings on the surface of the substrate, such as, for example, over or between the dots, electrodes, or cells. Such coatings can include, for example, silicone, an electrolytic mixture, hypoallergenic agents, drugs, biologics, stem cells, skin substitutes, cosmetic products, combinations thereof, or the like. Drugs suitable for use with embodiments of the invention include analgesics, antibiotics, anti-inflammatories, or the like. Embodiments can include multi-phase systems, for example wherein one array is on a substrate, and another array is suspended, for example in a gel, for example a hydrogel.

In certain embodiments that utilize a poly-cellulose binder, the binder itself can have a beneficial effect such as reducing the local concentration of matrix metallo-proteases through an iontophoretic process that drives the cellulose into the surrounding tissue. This process can be used to electronically drive other components such as drugs into the surrounding tissue.

The binder can comprise any biocompatible liquid material that can be mixed with a conductive element (preferably metallic crystals of silver or zinc) to create a conductive solution which can be applied to a substrate. One suitable binder is a solvent reducible polymer, such as the polyacrylic non-toxic silk-screen ink manufactured by COLORCON® Inc., a division of Berwind Pharmaceutical Services, Inc. (see COLORCON® NO-TOX product line, part number NT28). In an embodiment the binder is mixed with high purity (at least 99.99%, in an embodiment) metallic silver crystals to make the silver conductive solution. The silver crystals, which can be made by grinding silver into a powder, are preferably smaller than 100 microns in size or about as fine as flour. In an embodiment, the size of the crystals is about 325 mesh, which is typically about 40 microns in size or a little smaller. The binder is separately mixed with high purity (at least 99.99%, in an embodiment) metallic zinc powder which has also preferably been sifted through standard 325 mesh screen, to make the zinc conductive solution.

Other powders of metal can be used to make other conductive metal solutions in the same way as described in other embodiments.

When COLORCON® polyacrylic ink is used as the binder, about 10 to 40 percent of the mixture should be metal for a long term bandage (for example, one that stays on for about 10 days). For example, for a long term LLEC or LLEF system the percent of the mixture that should be metal can be 8 percent, or 10 percent, 12 percent, 14 percent, 16 percent, 18 percent, 20 percent, 22 percent, 24 percent, 26 percent, 28 percent, 30 percent, 32 percent, 34 percent, 36 percent, 38 percent, 40 percent, 42 percent, 44 percent, 46 percent, 48 percent, 50 percent, or the like.

If the same binder is used, but the percentage of the mixture that is metal is increased to 60 percent or higher, a typical system will be effective for longer. For example, for a longer term device, the percent of the mixture that should be metal can be 40 percent, 42 percent, 44 percent, 46 percent, 48 percent, 50 percent, 52 percent, 54 percent, 56 percent, 58 percent, 60 percent, 62 percent, 64 percent, 66 percent, 68 percent, 70 percent, 72 percent, 74 percent, 76 percent, 78 percent, 80 percent, 82 percent, 84 percent, 86 percent, 88 percent, 90 percent, or the like.

For systems comprising a pliable substrate it can be desired to decrease the percentage of metal down to 5 percent or less, or to use a binder that causes the crystals to be more deeply embedded, so that the primary surface will be antimicrobial for a very long period of time and will not wear prematurely. Other binders can dissolve or otherwise break down faster or slower than a polyacrylic ink, so adjustments can be made to achieve the desired rate of spontaneous reactions from the voltaic cells.

To maximize the number of voltaic cells, in various embodiments, a substrate can comprise a pattern of alternating silver masses (e.g., 6 as shown in FIG. 1) or electrodes or reservoirs and zinc masses (e.g., 10 as shown in FIG. 1) or electrodes or reservoirs can create an array of electrical currents. A basic embodiment, shown in FIG. 1, has each mass of silver randomly spaced from masses of zinc, and has each mass of zinc randomly spaced from masses of silver, according to an embodiment. In another embodiment, mass of silver can be equally spaced from masses of zinc, and have each mass of zinc equally spaced from masses of silver. That is, the electrodes or reservoirs or dots can either be a uniform pattern, a random pattern, or a combination of the like. The first electrode 6 is separated from the second electrode 10. The designs of first electrode 6 and second electrode 10 are simply round dots, and in an embodiment, are repeated throughout the hydrogel. For an exemplary device comprising silver and zinc, each silver design preferably has about twice as much mass as each zinc design, in an embodiment. For the embodiment in FIG. 1, the silver designs are preferably about a millimeter from each of the closest four zinc designs, and vice-versa. The resulting pattern of dissimilar metal masses defines an array of voltaic cells when introduced to an electrolytic solution. To maximize the density of electrical current over a primary surface the pattern of FIG. 2 can be used. The first electrode 6 in FIG. 2 is a large hexagonally shaped dot, and the second electrode 10 is a pair of smaller hexagonally shaped dots that are spaced from each other. The spacing 8 between the first electrode 6 and the second electrode 10 maintains a relatively consistent distance between adjacent sides of the designs. Numerous repetitions 12 of the designs result in a pattern 14 that can be described as at least one of the first design being surrounded by six hexagonally shaped dots of the second design.

Further, in FIG. 1, the dissimilar first electrode 6 and second electrode 10 are applied onto a desired primary surface 2 of an article 4. In one embodiment a primary surface is a surface of a system that comes into direct contact with an area to be treated such as a skin surface.

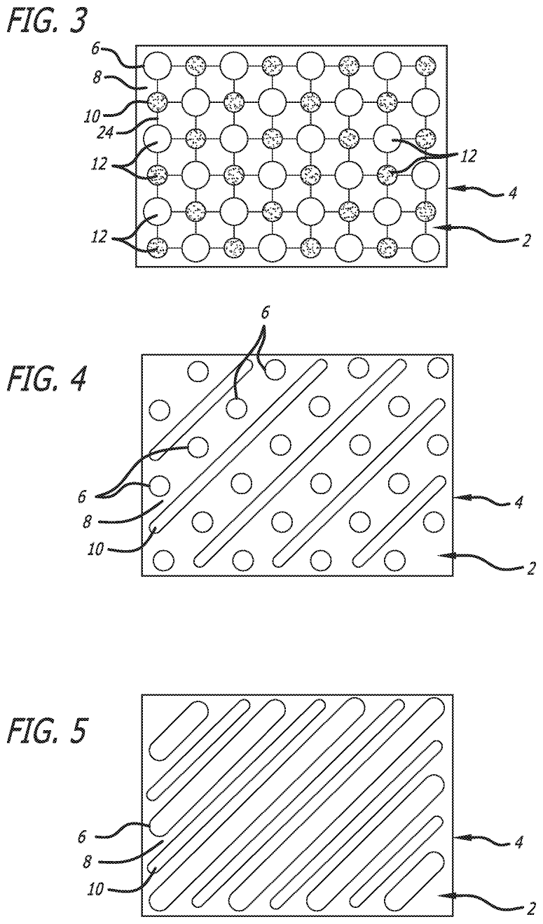

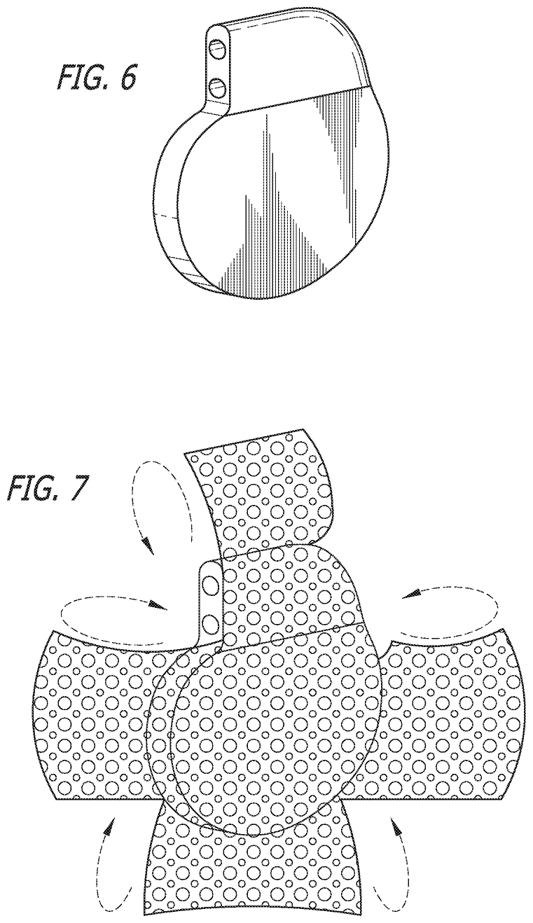

FIG. 3 shows an additional feature, which can be added between designs, that can initiate the flow of current in a poor electrolytic solution. A fine line 24 is printed using one of the conductive metal solutions along a current path of each voltaic cell. The fine line can initially have a direct reaction but will be depleted until the distance between the electrodes increases to where maximum voltage is realized. The initial current produced is intended to help control edema so that the system will be effective. If the electrolytic solution is highly conductive when the system is initially applied, the fine line can be quickly depleted and the device will function as though the fine line had never existed.

FIGS. 4 and 5 show alternative patterns that use at least one line design. The first electrode 6 of FIG. 4 is a round dot similar to the first design used in FIG. 1. The second electrode 10 of FIG. 4 is a line. When the designs are repeated, they define a pattern of parallel lines that are separated by numerous spaced dots. FIG. 5 uses only line designs. The first electrode 6 can be thicker or wider than the second electrode 10 if the oxidation-reduction reaction requires more metal from the first conductive element (mixed into the first design's conductive metal solution) than the second conductive element (mixed into the second design's conductive metal solution). The lines can be dashed. Another pattern can be silver grid lines that have zinc masses in the center of each of the cells of the grid. The pattern can be letters printed from alternating conductive materials so that a message can be printed onto the primary surface, for example a brand name or identifying information such as patient blood type.

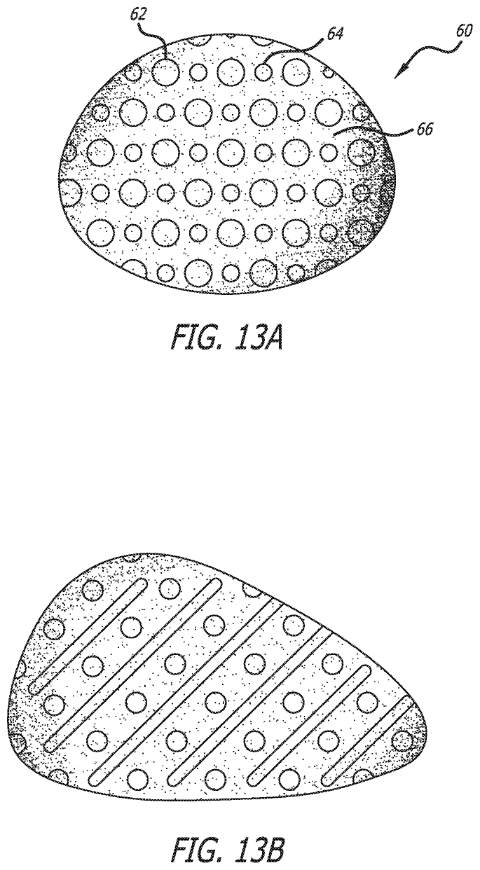

FIGS. 13A and 13B depict breast implants 60 comprising a multi-array matrix. This embodiment has each mass of silver randomly spaced from masses of zinc, and has each mass of zinc randomly spaced from masses of silver. In another embodiment, mass of silver can be equally spaced from masses of zinc, and have each mass of zinc equally spaced from masses of silver. That is, the electrodes or reservoirs or dots can either be a uniform pattern, a random pattern, or a combination of the like. The first electrode 62 is separated from the second electrode 64. The designs of first electrode 62 and second electrode 64 are simply round dots, and in an embodiment, are repeated throughout the implant surface. For an exemplary device comprising silver and zinc, each silver design preferably has about twice as much mass as each zinc design, in an embodiment. For the embodiment in FIGS. 13A and 13B, the silver designs are preferably about a millimeter from each of the closest four zinc designs, and vice-versa. The resulting pattern of dissimilar metal masses defines an array of voltaic cells when introduced to an electrolytic solution.

Because the spontaneous oxidation-reduction reaction of silver and zinc uses a ratio of approximately two silver to one zinc, the silver design can contain about twice as much mass as the zinc design in an embodiment. At a spacing of about 1 mm between the closest dissimilar metals (closest edge to closest edge) each voltaic cell that contacts a conductive fluid such as saline or a water based hydrogel can create approximately 1 volt of potential that will penetrate substantially through its surrounding surfaces. Closer spacing of the dots can reduce the strength of the electric field and the current will not penetrate as deeply. Therefore, spacing between the closest conductive materials can be, for example, 1 μm, 2 μm, 3 μm, 4 μm, 5 μm, 6 μm, 7 μm, 8 μm, 9 μm, 10 μm, 11 μm, 12 μm, 13 μm, 14 μm, 15 μm, 16 μm, 17 μm, 18 μm, 19 μm, 20 μm, 21 μm, 22 μm, 23 μm, 24 μm, 25 μm, 26 μm, 27 μm, 28 μm, 29 μm, 30 μm, 31 μm, 32 μm, 33 μm, 34 μm, 35 μm, 36 μm, 37 μm, 38 μm, 39 μm, 40 μm, 41 μm, 42 μm, 43 μm, 44 μm, 45 μm, 46 μm, 47 μm, 48 μm, 49 μm, 50 μm, 51 μm, 52 μm, 53 μm, 54 μm, 55 μm, 56 μm, 57 μm, 58 μm, 59 μm, 60 μm, 61 μm, 62 μm, 63 μm, 64 μm, 65 μm, 66 μm, 67 μm, 68 μm, 69 μm, 70 μm, 71 μm, 72 μm, 73 μm, 74 μm, 75 μm, 76 μm, 77 μm, 78 μm, 79 μm, 80 μm, 81 μm, 82 μm, 83 μm, 84 μm, 85 μm, 86 μm, 87 μm, 88 μm, 89 μm, 90 μm, 91 μm, 92 μm, 93 μm, 94 μm, 95 μm, 96 μm, 97 μm, 98 μm, 99 μm, 0.1 mm, or 0.2 mm, 0.3 mm, 0.4 mm, 0.5 mm, 0.6 mm, 0.7 mm, 0.8 mm, 0.9 mm, 1 mm, 1.1 mm, 1.2 mm, 1.3 mm, 1.4 mm, 1.5 mm, 1.6 mm, 1.7 mm, 1.8 mm, 1.9 mm, 2 mm, 2.1 mm, 2.2 mm, 2.3 mm, 2.4 mm, 2.5 mm, 2.6 mm, 2.7 mm, 2.8 mm, 2.9 mm, 3 mm, 3.1 mm, 3.2 mm, 3.3 mm, 3.4 mm, 3.5 mm, 3.6 mm, 3.7 mm, 3.8 mm, 3.9 mm, 4 mm, 4.1 mm, 4.2 mm, 4.3 mm, 4.4 mm, 4.5 mm, 4.6 mm, 4.7 mm, 4.8 mm, 4.9 mm, 5 mm, 5.1 mm, 5.2 mm, 5.3 mm, 5.4 mm, 5.5 mm, 5.6 mm, 5.7 mm, 5.8 mm, 5.9 mm, 6 mm, or the like.

In certain embodiments the spacing between the closest conductive materials can be not more than 1 μm, or not more than 2 μm, or not more than 3 μm, or not more than 4 μm, or not more than 5, or not more than 6 μm, or not more than 7 μm, or not more than 8 μm, or not more than 9 μm, or not more than 10 μm, or not more than 11 μm, or not more than 12 μm, or not more than 13 μm, or not more than 14 μm, or not more than 15 μm, or not more than 16 μm, or not more than 17 μm, or not more than 18 μm, or not more than 19 μm, or μm not more than 20 μm, or μm not more than 21 μm, or not more than 22 μm, or not more than 23 μm, or not more than 24 μm, or not more than 25 μm, or not more than 26 μm, or not more than 27 μm, or not more than 28 μm, or not more than 29 μm, or not more than 30 μm, or not more than 31 μm, or not more than 32 μm, or not more than 33 μm, or not more than 34 μm, or not more than 35 μm, or not more than 36 μm, or not more than 37 μm, or not more than 38 μm, or not more than 39 μm, or not more than 40 μm, or not more than 41 μm, or not more than 42 μm, or not more than 43 μm, or not more than 44 μm, or not more than 45 μm, or not more than 46 μm, or not more than 47 μm, or not more than 48 μm, or not more than 49 μm, or not more than 50 μm, or not more than 51 μm, or not more than 52 μm, or not more than 53 μm, or not more than 54 μm, or not more than 55 μm, or not more than 56 μm, or not more than 57 μm, or not more than 58 μm, or not more than 59 μm, or not more than 60 μm, or not more than 61 μm, or not more than 62 μm, or not more than 63 μm, or not more than 64 μm, or not more than 65 μm, or not more than 66 μm, or not more than 67 μm, or not more than 68 μm, or not more than 69 μm, or not more than 70 μm, or not more than 71 μm, or not more than 72 μm, or not more than 73 μm, or not more than 74 μm, or not more than 75 μm, or not more than 76 μm, or not more than 77 μm, or not more than 78 μm, or not more than 79 μm, or not more than 80 μm, or not more than 81 μm, or not more than 82 μm, or not more than 83 μm, or not more than 84 μm, or not more than 85 μm, or not more than 86 μm, or not more than 87 μm, or not more than 88 μm, or not more than 89 μm, or not more than 90 μm, or not more than 91 μm, or not more than 92 μm, or not more than 93 μm, or not more than 94 μm, or not more than 95 μm, or not more than 96 μm, or not more than 97 μm, or not more than 98 μm, or not more than 99 μm, not more than not more than 0.1 mm, not more than 0.2 mm, not more than 0.3 mm, not more than 0.4 mm, not more than 0.5 mm, not more than 0.6 mm, not more than 0.7 mm, not more than 0.8 mm, not more than 0.9 mm, not more than 1 mm, not more than 1.1 mm, not more than 1.2 mm, not more than 1.3 mm, not more than 1.4 mm, not more than 1.5 mm, not more than 1.6 mm, not more than 1.7 mm, not more than 1.8 mm, not more than 1.9 mm, not more than 2 mm, not more than 2.1 mm, not more than 2.2 mm, not more than 2.3 mm, not more than 2.4 mm, not more than 2.5 mm, not more than 2.6 mm, not more than 2.7 mm, not more than 2.8 mm, not more than 2.9 mm, not more than 3 mm, not more than 3.1 mm, not more than 3.2 mm, not more than 3.3 mm, not more than 3.4 mm, not more than 3.5 mm, not more than 3.6 mm, not more than 3.7 mm, not more than 3.8 mm, not more than 3.9 mm, not more than 4 mm, not more than 4.1 mm, not more than 4.2 mm, not more than 4.3 mm, not more than 4.4 mm, not more than 4.5 mm, not more than 4.6 mm, not more than 4.7 mm, not more than 4.8 mm, not more than 4.9 mm, not more than 5 mm, not more than 5.1 mm, not more than 5.2 mm, not more than 5.3 mm, not more than 5.4 mm, not more than 5.5 mm, not more than 5.6 mm, not more than 5.7 mm, not more than 5.8 mm, not more than 5.9 mm, not more than 6 mm, or the like.

In certain embodiments spacing between the closest conductive materials can be not less than 1 μm, or not less than 2 μm, or not less than 3 μm, or not less than 4 μm, or not less than 5 μm, or not less than 6 μm, or not less than 7 μm, or not less than 8 μm, or not less than 9 μm, or not less than 10 μm, or not less than 11 μm, or not less than 12 μm, or not less than 13 μm, or not less than 14 μm, or not less than 15 μm, or not less than 16 μm, or not less than 17 μm, or not less than 18 μm, or not less than 19 μm, or not less than 20 μm, or not less than 21 μm, or not less than 22 μm, or not less than 23 μm, or not less than 24 μm, or not less than 25 μm, or not less than 26 μm, or not less than 27 μm, or not less than 28 μm, or not less than 29 μm, or not less than 30 μm, or not less than 31 μm, or not less than 32 μm, or not less than 33 μm, or not less than 34 μm, or not less than 35 μm, or not less than 36 μm, or not less than 37 μm, or not less than 38 μm, or not less than 39 μm, or not less than 40 μm, or not less than 41 μm, or not less than 42 μm, or not less than 43 μm, or not less than 44 μm, or not less than 45 μm, or not less than 46 μm, or not less than 47 μm, or not less than 48 μm, or not less than 49 μm, or not less than 50 μm, or not less than 51 μm, or not less than 52 μm, or not less than 53 μm, or not less than 54 μm, or not less than 55 μm, or not less than 56 μm, or not less than 57 μm, or not less than 58 μm, or not less than 59 μm, or not less than 60 μm, or not less than 61 μm, or not less than 62 μm, or not less than 63 μm, or not less than 64 μm, or not less than 65 μm, or not less than 66 μm, or not less than 67 μm, or not less than 68 μm, or not less than 69 μm, or not less than 70 μm, or not less than 71 μm, or not less than 72 μm, or not less than 73 μm, or not less than 74 μm, or not less than 75 μm, or not less than 76 μm, or not less than 77 μm, or not less than 78 μm, or not less than 79 μm, or not less than 80 μm, or not less than 81 μm, or not less than 82 μm, or not less than 83 μm, or not less than 84 μm, or not less than 85 μm, or not less than 86 μm, or not less than 87 μm, or not less than 88 μm, or not less than 89 μm, or not less than 90 μm, or not less than 91 μm, or not less than 92 μm, or not less than 93 μm, or not less than 94 μm, or not less than 95 μm, or not less than 96 μm, or not less than 97 μm, or not less than 98 μm, or not less than 99 μm, or not less than 0.1 mm, not less than 0.2 mm, not less than 0.3 mm, not less than 0.4 mm, not less than 0.5 mm, not less than 0.6 mm, not less than 0.7 mm, not less than 0.8 mm, not less than 0.9 mm, not less than 1 mm, not less than 1.1 mm, not less than 1.2 mm, not less than 1.3 mm, not less than 1.4 mm, not less than 1.5 mm, not less than 1.6 mm, not less than 1.7 mm, not less than 1.8 mm, not less than 1.9 mm, not less than 2 mm, not less than 2.1 mm, not less than 2.2 mm, not less than 2.3 mm, not less than 2.4 mm, not less than 2.5 mm, not less than 2.6 mm, not less than 2.7 mm, not less than 2.8 mm, not less than 2.9 mm, not less than 3 mm, not less than 3.1 mm, not less than 3.2 mm, not less than 3.3 mm, not less than 3.4 mm, not less than 3.5 mm, not less than 3.6 mm, not less than 3.7 mm, not less than 3.8 mm, not less than 3.9 mm, not less than 4 mm, not less than 4.1 mm, not less than 4.2 mm, not less than 4.3 mm, not less than 4.4 mm, not less than 4.5 mm, not less than 4.6 mm, not less than 4.7 mm, not less than 4.8 mm, not less than 4.9 mm, not less than 5 mm, not less than 5.1 mm, not less than 5.2 mm, not less than 5.3 mm, not less than 5.4 mm, not less than 5.5 mm, not less than 5.6 mm, not less than 5.7 mm, not less than 5.8 mm, not less than 5.9 mm, not less than 6 mm, or the like.

Embodiments comprise systems and devices comprising a hydrophilic polymer base and a first electrode design formed from a first conductive liquid that comprises a mixture of a polymer and a first element, the first conductive liquid being applied into a position of contact with the primary surface, the first element comprising a metal species, and the first electrode design comprising at least one dot or reservoir, wherein selective ones of the at least one dot or reservoir have approximately a 1.5 mm+/−1 mm mean diameter; a second electrode design formed from a second conductive liquid that comprises a mixture of a polymer and a second element, the second element comprising a different metal species than the first element, the second conductive liquid being printed into a position of contact with the primary surface, and the second electrode design comprising at least one other dot or reservoir, wherein selective ones of the at least one other dot or reservoir have approximately a 2 mm+/−2 mm mean diameter; a spacing on the primary surface that is between the first electrode design and the second electrode design such that the first electrode design does not physically contact the second electrode design, wherein the spacing is approximately 1.5 mm+/−1 mm, and at least one repetition of the first electrode design and the second electrode design, the at least one repetition of the first electrode design being substantially adjacent the second electrode design, wherein the at least one repetition of the first electrode design and the second electrode design, in conjunction with the spacing between the first electrode design and the second electrode design, defines at least one pattern of at least one voltaic cell for spontaneously generating at least one electrical current when introduced to an electrolytic solution. Therefore, electrodes, dots, or reservoirs can have a mean diameter of 0.1 mm, 0.2 mm, 0.3 mm, 0.4 mm, 0.5 mm, 0.6 mm, 0.7 mm, 0.8 mm, 0.9 mm, 1.0 mm, 1.1 mm, 1.2 mm, 1.3 mm, 1.4 mm, 1.5 mm, 1.6 mm, 1.7 mm, 1.8 mm, 1.9 mm, 2.0 mm, 2.1 mm, 2.2 mm, 2.3 mm, 2.4 mm, 2.5 mm, 2.6 mm, 2.7 mm, 2.8 mm, 2.9 mm, 3.0 mm, 3.1 mm, 3.2 mm, 3.3 mm, 3.4 mm, 3.5 mm, 3.6 mm, 3.7 mm, 3.8 mm, 3.9 mm, 4.0 mm, 4.1 mm, 4.2 mm, 4.3 mm, 4.4 mm, 4.5 mm, 4.6 mm, 4.7 mm, 4.8 mm, 4.9 mm, 5.0 mm, or the like.

In further embodiments, electrodes, dots or reservoirs can have a mean diameter of not less than 0.1 mm, not less than 0.2 mm, not less than 0.3 mm, not less than 0.4 mm, not less than 0.5 mm, not less than 0.6 mm, not less than 0.7 mm, not less than 0.8 mm, not less than 0.9 mm, not less than 1.0 mm, not less than 1.1 mm, not less than 1.2 mm, not less than 1.3 mm, not less than 1.4 mm, not less than 1.5 mm, not less than 1.6 mm, not less than 1.7 mm, not less than 1.8 mm, not less than 1.9 mm, not less than 2.0 mm, not less than 2.1 mm, not less than 2.2 mm, not less than 2.3 mm, not less than 2.4 mm, not less than 2.5 mm, not less than 2.6 mm, not less than 2.7 mm, not less than 2.8 mm, not less than 2.9 mm, not less than 3.0 mm, not less than 3.1 mm, not less than 3.2 mm, not less than 3.3 mm, not less than 3.4 mm, not less than 3.5 mm, not less than 3.6 mm, not less than 3.7 mm, not less than 3.8 mm, not less than 3.9 mm, not less than 4.0 mm, not less than 4.1 mm, not less than 4.2 mm, not less than 4.3 mm, not less than 4.4 mm, not less than 4.5 mm, not less than 4.6 mm, not less than 4.7 mm, not less than 4.8 mm, not less than 4.9 mm, not less than 5.0 mm, or the like.

In further embodiments, electrodes, dots or reservoirs can have a mean diameter of not more than 0.1 mm, not more than 0.2 mm, or not more than 0.3 mm, not more than 0.4 mm, not more than 0.5 mm, not more than 0.6 mm, not more than 0.7 mm, not more than 0.8 mm, not more than 0.9 mm, not more than 1.0 mm, not more than 1.1 mm, not more than 1.2 mm, not more than 1.3 mm, not more than 1.4 mm, not more than 1.5 mm, not more than 1.6 mm, not more than 1.7 mm, not more than 1.8 mm, not more than 1.9 mm, not more than 2.0 mm, not more than 2.1 mm, not more than 2.2 mm, not more than 2.3 mm, not more than 2.4 mm, not more than 2.5 mm, not more than 2.6 mm, not more than 2.7 mm, not more than 2.8 mm, not more than 2.9 mm, not more than 3.0 mm, not more than 3.1 mm, not more than 3.2 mm, not more than 3.3 mm, not more than 3.4 mm, not more than 3.5 mm, not more than 3.6 mm, not more than 3.7 mm, not more than 3.8 mm, not more than 3.9 mm, not more than 4.0 mm, not more than 4.1 mm, not more than 4.2 mm, not more than 4.3 mm, not more than 4.4 mm, not more than 4.5 mm, not more than 4.6 mm, not more than 4.7 mm, not more than 4.8 mm, not more than 4.9 mm, not more than 5.0 mm, or the like.

In further embodiments, electrodes, dots or reservoirs can have a mean radius of not less than 0.1 mm, not less than 0.2 mm, not less than 0.3 mm, not less than 0.4 mm, not less than 0.5 mm, not less than 0.6 mm, not less than 0.7 mm, not less than 0.8 mm, not less than 0.9 mm, not less than 1.0 mm, not less than 1.1 mm, not less than 1.2 mm, not less than 1.3 mm, not less than 1.4 mm, not less than 1.5 mm, not less than 1.6 mm, not less than 1.7 mm, not less than 1.8 mm, not less than 1.9 mm, not less than 2.0 mm, not less than 2.1 mm, not less than 2.2 mm, not less than 2.3 mm, not less than 2.4 mm, not less than 2.5 mm, not less than 2.6 mm, not less than 2.7 mm, not less than 2.8 mm, not less than 2.9 mm, not less than 3.0 mm, not less than 3.1 mm, not less than 3.2 mm, not less than 3.3 mm, not less than 3.4 mm, not less than 3.5 mm, not less than 3.6 mm, not less than 3.7 mm, not less than 3.8 mm, not less than 3.9 mm, not less than 4.0 mm, not less than 4.1 mm, not less than 4.2 mm, not less than 4.3 mm, not less than 4.4 mm, not less than 4.5 mm, not less than 4.6 mm, not less than 4.7 mm, not less than 4.8 mm, not less than 4.9 mm, not less than 5.0 mm, or the like.

In further embodiments, electrodes, dots or reservoirs can have a mean radius of not more than 0.1 mm, not more than 0.2 mm, or not more than 0.3 mm, not more than 0.4 mm, not more than 0.5 mm, not more than 0.6 mm, not more than 0.7 mm, not more than 0.8 mm, not more than 0.9 mm, not more than 1.0 mm, not more than 1.1 mm, not more than 1.2 mm, not more than 1.3 mm, not more than 1.4 mm, not more than 1.5 mm, not more than 1.6 mm, not more than 1.7 mm, not more than 1.8 mm, not more than 1.9 mm, not more than 2.0 mm, not more than 2.1 mm, not more than 2.2 mm, not more than 2.3 mm, not more than 2.4 mm, not more than 2.5 mm, not more than 2.6 mm, not more than 2.7 mm, not more than 2.8 mm, not more than 2.9 mm, not more than 3.0 mm, not more than 3.1 mm, not more than 3.2 mm, not more than 3.3 mm, not more than 3.4 mm, not more than 3.5 mm, not more than 3.6 mm, not more than 3.7 mm, not more than 3.8 mm, not more than 3.9 mm, not more than 4.0 mm, not more than 4.1 mm, not more than 4.2 mm, not more than 4.3 mm, not more than 4.4 mm, not more than 4.5 mm, not more than 4.6 mm, not more than 4.7 mm, not more than 4.8 mm, not more than 4.9 mm, not more than 5.0 mm, or the like.

The material concentrations or quantities within and/or the relative sizes (e.g., dimensions or surface area) of the first and second reservoirs or dots or electrodes can be selected deliberately to achieve various characteristics of the systems' behavior. For example, the quantities of material within a first and second reservoir can be selected to provide an apparatus having an operational behavior that depletes at approximately a desired rate and/or that “dies” after an approximate period of time after activation, for example at a similar pace as the resorption of the substrate, or faster than the resorption of the substrate. In an embodiment the one or more first reservoirs and the one or more second reservoirs are configured to sustain one or more currents for an approximate predetermined period of time, after activation. It is to be understood that the amount of time that currents are sustained can depend on external conditions and factors (e.g., the quantity and type of activation material), and currents can occur intermittently depending on the presence or absence of activation material.

In a particular embodiment the difference of the standard potentials of the first and second reservoirs can be at least 0.05 V, at least 0.06 V, at least 0.07 V, at least 0.08 V, at least 0.09 V, at least 0.1 V, at least 0.2 V, at least 0.3 V, at least 0.4 V, at least 0.5 V, at least 0.6 V, at least 0.7 V, at least 0.8 V, at least 0.9 V, at least 1.0 V, at least 1.1 V, at least 1.2 V, at least 1.3 V, at least 1.4 V, at least 1.5 V, at least 1.6 V, at least 1.7 V, at least 1.8 V, at least 1.9 V, at least 2.0 V, at least 2.1 V, at least 2.2 V, at least 2.3 V, at least 2.4 V, at least 2.5 V, at least 2.6 V, at least 2.7 V, at least 2.8 V, at least 2.9 V, at least 3.0 V, at least 3.1 V, at least 3.2 V, at least 3.3 V, at least 3.4 V, at least 3.5 V, at least 3.6 V, at least 3.7 V, at least 3.8 V, at least 3.9 V, at least 4.0 V, at least 4.1 V, at least 4.2 V, at least 4.3 V, at least 4.4 V, at least 4.5 V, at least 4.6 V, at least 4.7 V, at least 4.8 V, at least 4.9 V, at least 5.0 V, or the like.

In a particular embodiment, the difference of the standard potentials of the first and second reservoirs can be not more than 0.05 V, not more than 0.06 V, not more than 0.07 V, not more than 0.08 V, not more than 0.09 V, not more than 0.1 V, not more than 0.2 V, not more than 0.3 V, not more than 0.4 V, not more than 0.5 V, not more than 0.6 V, not more than 0.7 V, not more than 0.8 V, not more than 0.9 V, not more than 1.0 V, not more than 1.1 V, not more than 1.2 V, not more than 1.3 V, not more than 1.4 V, not more than 1.5 V, not more than 1.6 V, not more than 1.7 V, not more than 1.8 V, not more than 1.9 V, not more than 2.0 V, not more than 2.1 V, not more than 2.2 V, not more than 2.3 V, not more than 2.4 V, not more than 2.5 V, not more than 2.6 V, not more than 2.7 V, not more than 2.8 V, not more than 2.9 V, not more than 3.0 V, not more than 3.1 V, not more than 3.2 V, not more than 3.3 V, not more than 3.4 V, not more than 3.5 V, not more than 3.6 V, not more than 3.7 V, not more than 3.8 V, not more than 3.9 V, not more than 4.0 V, not more than 4.1 V, not more than 4.2 V, not more than 4.3 V, not more than 4.4 V, not more than 4.5 V, not more than 4.6 V, not more than 4.7 V, not more than 4.8 V, not more than 4.9 V, not more than 5.0 V, or the like. In embodiments that include very small reservoirs (e.g., on the nanometer scale), the difference of the standard potentials can be substantially less or more. The electrons that pass between the first reservoir and the second reservoir can be generated as a result of the difference of the standard potentials.

The voltage present at the site of use of the system or device is typically in the range of millivolts, but disclosed embodiments can introduce a much higher voltage, for example near 1 volt when using the 1 mm spacing of dissimilar metals already described. In this way the current not only can drive silver and zinc into the treatment if desired for treatment, but the current can also provide a stimulatory current so that the entire surface area can be treated. The electric field can also have beneficial effects on cell migration, ATP production, and angiogenesis.

Certain embodiments are designed for universal conformability with any area of the exterior or interior of the body, for example a flat area or a contoured area. In embodiments the dressings or substrates are configured to conform to the area to be treated, for example by producing the dressing or substrate in particular shapes including “slits” or discontinuous regions. In embodiments the dressing or substrate can be produced in a U shape wherein the “arms” of the U are substantially equal in length as compared to the “base” of the U. In embodiments the dressing can be produced in a U shape wherein the “arms” of the U are substantially longer in length as compared to the “base” of the U. In embodiments the dressing can be produced in a U shape wherein the “arms” of the U are substantially shorter in length as compared to the “base” of the U. In embodiments the substrate can be produced in an X shape wherein the “arms” of the X are substantially equal in length.

The systems and devices can comprise corresponding or interlocking perimeter areas to assist the devices in maintaining their position on or in the patient and/or their position relative to each other. In certain embodiments, the systems and devices can comprise a port or ports to provide access to the treatment area beneath the device.

In embodiments the system can comprise a port to access the interior of the substrate, for example to add hydration, active agents, carriers, solvents, or some other material.

In further embodiments the device can comprise a wire to electrically link the device with other components, such as monitoring equipment or a power source. In embodiments the device can be wirelessly linked to monitoring or data collection equipment, for example linked via Bluetooth to a cell phone or computer that collects data from the device. In certain embodiments the device can comprise data collection means, such as temperature, pH, pressure, or conductivity data collection means.

In embodiments the device can be shaped to fit an area of desired use, for example a surgical incision.

Disclosed embodiments can comprise multiple-piece dressings. For example, embodiments comprise multiple piece dressings that “interlock” across the application site, such as a wound. For example, embodiments can comprise one element of a two-piece dressing for application on one side of a surgical incision, and the second element of a two-piece dressing for application on the opposite side of the incision.

In embodiments the system or device can comprise additional materials to aid in treatment.

In embodiments, the system or device can comprise instructions or directions on how to place the system to maximize its performance. Embodiments comprise a kit comprising a system and directions for its use.

In certain embodiments dissimilar metals can be used to create an electric field with a desired voltage within the device or system. In certain embodiments the pattern of reservoirs can control the watt density and shape of the electric field.

Certain embodiments can utilize a power source to create the electric current, such as a battery or a micro-battery. The power source can be any energy source capable of generating a current in the system and can comprise, for example, AC power, DC power, radio frequencies (RF) such as pulsed RF, induction, ultrasound, and the like.

Dissimilar metals used to make a system or device disclosed herein can be, for example, silver and zinc. In certain embodiments, the electrodes are coupled with a non-conductive material to create a random dot pattern or a uniform dot pattern within a hydrogel, most preferably an array or multi-array of voltaic cells that do not spontaneously react until they contact an electrolytic solution. Sections of this description use the terms “coated,” “plated,” or “printed” with “ink,” but it is to be understood that a “dot” or “bead” in a hydrogel can also be a solid microsphere of conductive material. The use of any suitable means for applying a conductive material is contemplated. In embodiments “coated,” “plated,” or “printed” can comprise any material such as a solution suitable for forming an electrode on a surface of a microsphere such as a conductive material comprising a conductive metal solution.

In another embodiment, “coated,” “plated,” or “printed” can comprise electroplating microspheres. Electroplating is a process that uses electric current to reduce dissolved metal cations so that they form a coherent metal coating on an electrode. Electroplating can be used to change the surface properties of microspheres or to build up thickness of a microsphere. Building thickness by electroplating microspheres can allow the microspheres to be form with a specific conductive material and at a specific gravity determined by the user.

In embodiments, printing devices can be used to produce systems and devices as disclosed herein. For example, inkjet or “3D” printers can be used to produce embodiments. In certain embodiments the binders or inks used to produce iontophoresis systems disclosed herein can comprise, for example, poly cellulose inks, poly acrylic inks, poly urethane inks, silicone inks, and the like. In embodiments the type of ink used can determine the release rate of electrons from the reservoirs. In embodiments various materials can be added to the ink or binder such as, for example, conductive or resistive materials can be added to alter the shape or strength of the electric field. Other materials, such as silicon, can be added, for example, to reduce scar formation. Such materials can also be added to the spaces between reservoirs.

Dissimilar metals used to make a LLEC or LLEF system disclosed herein can be silver and zinc, and the electrolytic solution can include sodium chloride in water. In certain embodiments the electrodes are applied onto a non-conductive surface to create a pattern, most preferably an array or multi-array of voltaic cells that do not spontaneously react until they contact an electrolytic solution. Sections of this description use the terms “printing” with “ink,” but it is to be understood that the patterns may also be “painted” with “paints.” The use of any suitable means for applying a conductive material is contemplated. In embodiments “ink” or “paint” can comprise any material such as a solution suitable for forming an electrode on a surface such as a conductive material including a conductive metal solution. In embodiments “printing” or “painted” can comprise any method of applying a solution to a material upon which a matrix is desired.

In certain embodiments, the system or device can be shaped to fit a particular region of the body.

Embodiments disclosed herein can comprise interlocking areas on the perimeter of that complement other areas on the perimeter such that the areas engage with each other by the fitting together of projections or protrusions and recesses or intrusions. Such embodiments provide several advantages, for example additional securing force for the device, as well as allowing a user to custom-fit the device over a specific area. Multiple devices can be linked together to provide complete coverage over or inside wounds or incisions that are typically difficult to cover with a single device.

Certain embodiments disclosed herein include a method of manufacturing a LLEC or LLEF system, the method comprising joining with a substrate multiple first reservoirs wherein selected ones of the multiple first reservoirs include a reducing agent, and wherein first reservoir surfaces of selected ones of the multiple first reservoirs are proximate to a first substrate surface; and joining with the substrate multiple second reservoirs wherein selected ones of the multiple second reservoirs include an oxidizing agent, and wherein second reservoir surfaces of selected ones of the multiple second reservoirs are proximate to the first substrate surface, wherein joining the multiple first reservoirs and joining the multiple second reservoirs comprises joining using tattooing. In embodiments the substrate can comprise gauzes comprising dots or electrodes.

Further embodiments can include a method of manufacturing a LLEC or LLEF system, the method comprising joining with a substrate multiple first reservoirs wherein selected ones of the multiple first reservoirs include a reducing agent, and wherein first reservoir surfaces of selected ones of the multiple first reservoirs are proximate to a first substrate surface; and joining with the substrate multiple second reservoirs wherein selected ones of the multiple second reservoirs include an oxidizing agent, and wherein second reservoir surfaces of selected ones of the multiple second reservoirs are proximate to the first substrate surface, wherein joining the multiple first reservoirs and joining the multiple second reservoirs comprises: combining the multiple first reservoirs, the multiple second reservoirs, and multiple parallel insulators to produce a pattern repeat arranged in a first direction across a plane, the pattern repeat including a sequence of a first one of the parallel insulators, one of the multiple first reservoirs, a second one of the parallel insulators, and one of the multiple second reservoirs; and weaving multiple transverse insulators through the first parallel insulator, the one first reservoir, the second parallel insulator, and the one second reservoir in a second direction across the plane to produce a woven apparatus.

Embodiments disclosed herein comprise systems that can produce an electrical stimulus and/or can electromotivate, electroconduct, electroinduct, electrotransport, and/or electrophorese one or more therapeutic materials in areas of target tissue (e.g., iontophoresis).

In certain embodiments, for example treatment methods, it can be preferable to utilize AC or DC current. For example, embodiments disclosed herein can employ phased array, pulsed, square wave, sinusoidal, or other wave forms, combinations, or the like. Certain embodiments utilize a controller to produce and control power production and/or distribution to the device.

Embodiments disclosed herein relating to treatment can also comprise selecting a patient or tissue in need of, or that could benefit by, using a disclosed system.

While various embodiments have been shown and described, it will be realized that alterations and modifications can be made thereto without departing from the scope of the following claims. It is expected that other methods of applying the conductive material can be substituted as appropriate. Also, there are numerous shapes, sizes and patterns of voltaic cells that have not been described but it is expected that this disclosure will enable those skilled in the art to incorporate their own designs which will then which will become active when brought into contact with an electrolytic solution.

Aspects disclosed herein include systems, devices, and methods for data collection and/or data transmission, for example using bioelectric devices that comprise a resorbable substrate with one or more sensing elements, multi-array matrix of biocompatible microcells which can generate a LLEF or LLEC, and wherein a data element is collected from the sensing element and transmitted by a control module to a external device. Embodiments can include, for example, data collection equipment so as to track and/or quantify a user's movements or performance. Embodiments can include, for example, an accelerometer, so as to measure a user's speed, or impact forces on a user. Embodiments can include optical data collection devices, for example a camera.

In embodiments the device can be mechanically or wirelessly linked to monitoring or data collection equipment, for example linked via Bluetooth to a cell phone or computer that collects data from the device. In certain embodiments, disclosed devices and systems can comprise data collection means, such as location, temperature, pH, pressure, or conductivity data collection means. Embodiments can comprise a display, for example to visually present, for example, the location, temperature, pH, pressure, or conductivity data to a user.

In embodiments, the visual display can indicate when a data reading is outside a desired or approved range. For example, in an embodiment the device can provide a visual or audible warning or alarm when an accelerometer reading indicates an impact greater than the desired range, or a visual or audible warning or alarm when a temperature, pulse, or respiration reading is outside a desired range.

Methods of Use

Methods disclosed herein can comprise applying a disclosed embodiment to an area to be treated. Embodiments can comprise selecting or identifying a patient in need of treatment. In embodiments, methods disclosed herein can comprise formation and application of a system or device disclosed herein to an area to be treated. For example, in embodiments, a device to be implanted into a mammal, for example a pacemaker, can be “taped” or “pouched” with a disclosed embodiment.

For example, disclosed methods can comprise breast enhancement or reconstruction, for example, using any of an inframammary incision, a periareolar incision, a transaxillary incision, a transumbilical incision, a transabdominal incision, or combinations thereof.

Disclosed methods can comprise subglandular emplacement of a breast implant, wherein the breast implant is emplaced to the retromammary space, between the breast tissue (the mammary gland) and the pectoralis major muscle (major muscle of the chest), which most approximates the plane of normal breast tissue, and affords the most aesthetic results.

Disclosed methods can comprise subfascial emplacement of a breast implant, wherein the breast implant is emplaced beneath the fascia of the pectoralis major muscle; the subfascial position is a variant of the subglandular position for the breast implant. The technical advantages of the subfascial implant-pocket technique are debated; proponent surgeons report that the layer of fascial tissue provides greater implant coverage and better sustains its position.

Disclosed embodiments can comprise subpectoral (dual plane) emplacement of a breast implant, wherein the breast implant is emplaced beneath the pectoralis major muscle, after the surgeon releases the inferior muscular attachments, with or without partial dissection of the subglandular plane. Resultantly, the upper pole of the implant is partially beneath the pectoralis major muscle, while the lower pole of the implant is in the subglandular plane. This implantation technique achieves maximal coverage of the upper pole of the implant, whilst allowing the expansion of the implant's lower pole; however, “animation deformity”, the movement of the implants in the subpectoral plane can be excessive for some patients.

Disclosed embodiments can comprise submuscular emplacement of a breast implant, wherein the breast implant is emplaced beneath the pectoralis major muscle, without releasing the inferior origin of the muscle proper. Total muscular coverage of the implant can be achieved by releasing the lateral muscles of the chest wall-either the serratus muscle or the pectoralis minor muscle, or both—and suturing it, or them, to the pectoralis major muscle. In breast reconstruction surgery, the submuscular implantation approach effects maximal coverage of the breast implants. This technique is rarely used in cosmetic surgery due to high risk of animation deformities.

Methods disclosed herein include LLEC and LLEF systems that can promote and/or accelerate, for example, wound healing, the muscle recovery process, and the like.

Methods disclosed herein can increase intracellular calcium levels by exposing cells to the electric field produced by disclosed embodiments.

Further embodiments disclosed herein can direct cell migration.

Further embodiments can increase cellular protein sulfhydryl levels and cellular glucose uptake. Increased glucose uptake can result in greater mitochondrial activity and thus increased glucose utilization.

Disclosed embodiments can accelerate would healing, for example by activating enzymes that aid in the muscle recovery process, increasing glucose uptake, driving redox signaling, increasing H₂O₂ production, increasing cellular protein sulfhydryl levels, and increasing (IGF)-1 R phosphorylation.

Disclosed embodiments can prevent or repair muscle damage (for example such as can occur during a workout), for example by activating enzymes that aid in the muscle recovery process, increasing glucose uptake, driving redox signaling, increasing H₂O₂ production, increasing cellular protein sulfhydryl levels, and increasing (IGF)-1 R phosphorylation.

Disclosed methods can comprise the application of multiple-piece dressings. For example, embodiments comprise multiple piece dressings that “interlock” across or inside the application site, such as a wound or incision. Embodiments can comprise application of one element of a two-piece dressing for application on one side of an incision, and application of the second element of a two-piece dressing on the opposite side of the incision.

In embodiments, disclosed methods comprise application to the treatment area or the device of a system disclosed herein comprising an active agent. In embodiments the active agent can be, for example, positively or negatively charged. In embodiments, positively charged active agents can comprise centbucridine, tetracaine, Novocaine® (procaine), ambucaine, amolanone, amylcaine, benoxinate, betoxycaine, carticaine, chloroprocaine, cocaethylene, cyclomethycaine, butethamine, butoxycaine, carticaine, dibucaine, dimethisoquin, dimethocaine, diperodon, dyclonine, ecogonidine, ecognine, euprocin, fenalcomine, formocaine, hexylcaine, hydroxyteteracaine, leucinocaine, levoxadrol, metabutoxycaine, myrtecaine, butamben, bupivicaine, mepivacaine, beta-adrenoceptor antagonists, opioid analgesics, butanilicaine, ethyl aminobenzoate, fomocine, hydroxyprocaine, isobutyl p-aminobenzoate, naepaine, octacaine, orthocaine, oxethazaine, parenthoxycaine, phenacine, phenol, piperocaine, polidocanol, pramoxine, prilocalne, propanocaine, proparacaine, propipocaine, pseudococaine, pyrrocaine, salicyl alcohol, parethyoxycaine, piridocaine, risocaine, tolycaine, trimecaine, tetracaine, anticonvulsants, antihistamines, articaine, cocaine, procaine, amethocaine, chloroprocaine, marcaine, chloroprocaine, etidocaine, prilocaine, lignocaine, benzocaine, zolamine, ropivacaine, and dibucaine, dexamethasone phosphate, combinations thereof.

In embodiments, disclosed methods include application to the treatment area or the device of an antibacterial. In embodiments the antibacterial can be, for example, alcohols, aldehydes, halogen-releasing compounds, peroxides, anilides, biguanides, bisphenols, halophenols, heavy metals, phenols and cresols, quaternary ammonium compounds, and the like. In embodiments the antibacterial agent can comprise, for example, ethanol, isopropanol, glutaraldehyde, formaldehyde, chlorine compounds, iodine compounds, hydrogen peroxide, ozone, peracetic acid, formaldehyde, ethylene oxide, triclocarban, chlorhexidine, alexidine, polymeric biguanides, triclosan, hexachlorophene, PCMX (p-chloro-m-xylenol), silver compounds, mercury compounds, phenol, cresol, cetrimide, benzalkonium chloride, cetylpyridinium chloride, ceftolozane/tazobactam, ceftazidime/avibactam, ceftaroline/avibactam, imipenem/MK-7655, plazomicin, eravacycline, brilacidin, and the like.

In embodiments, compounds that modify resistance to common antibacterials can be employed. For example, some resistance-modifying agents may inhibit multidrug resistance mechanisms, such as drug efflux from the cell, thus increasing the susceptibility of bacteria to an antibacterial. In embodiments, these compounds can include Phe-Arg-β-naphthylamide, or β-lactamase inhibitors such as clavulanic acid and sulbactam.

### EXAMPLES

The following non-limiting example is provided for illustrative purposes only in order to facilitate a more complete understanding of representative embodiments. This example should not be construed to limit any of the embodiments described in the present specification.

### Example 1

### Cell Migration Assay

The in vitro scratch assay is an easy, low-cost and well-developed method to measure cell migration in vitro. The basic steps involve creating a “scratch” in a cell monolayer, capturing images at the beginning and at regular intervals during cell migration to close the scratch, and comparing the images to quantify the migration rate of the cells. Compared to other methods, the in vitro scratch assay is particularly suitable for studies on the effects of cell-matrix and cell-cell interactions on cell migration, mimic cell migration during wound healing in vivo and are compatible with imaging of live cells during migration to monitor intracellular events if desired. In addition to monitoring migration of homogenous cell populations, this method has also been adopted to measure migration of individual cells in the leading edge of the scratch. Not taking into account the time for transfection of cells, in vitro scratch assay per se usually takes from several hours to overnight.

Human keratinocytes were plated under plated under placebo or a LLEC system (substrate layer as described herein; labeled “PROCELLERA®”). Cells were also plated under silver-only or zinc-only dressings. After 24 hours, the scratch assay was performed. Cells plated under the PROCELLERA® device displayed increased migration into the “scratched” area as compared to any of the zinc, silver, or placebo dressings. After 9 hours, the cells plated under the PROCELLERA® device had almost “closed” the scratch. This demonstrates the importance of electrical activity to cell migration and infiltration.

In addition to the scratch test, genetic expression was tested. Increased insulin growth factor (IGF)-1R phosphorylation was demonstrated by the cells plated under the PROCELLERA® device as compared to cells plated under insulin growth factor alone.

Integrin accumulation also affects cell migration. An increase in integrin accumulation was achieved with the LLEC system. Integrin is necessary for cell migration, and is found on the leading edge of migrating cell.

Thus, the tested LLEC system enhanced cellular migration and IGF-1R/integrin involvement. This involvement demonstrates the effect that the LLEC system had upon cell receptors involved with the wound healing process.

### Example 2

### Zone of Inhibition Test

For cellular repair to be most efficient, available energy should not be shared with ubiquitous microbes. In this “zone of inhibition” test, placebo, a LLEC device (substrate layer as described herein; PROCELLERA®) and silver only were tested in an agar medium with a 24 hour growth of organisms. Bacteria grew over the placebo, there was a zone of inhibition over the PROCELLERA® and a minimal inhibition zone over the silver. Because the samples were “buried” in agar, the electricidal effect of the LLEC system could be tested. This could mean the microbes were affected by the electrical field or the silver ion transport through the agar was enhanced in the presence of the electric field. Silver ion diffusion, the method used by silver based antimicrobials, alone was not sufficient. The test demonstrates the improved bactericidal effect of PROCELLERA® as compared to silver alone.

### Example 3

### Wound Care Study

The medical histories of patients who received “standard-of-care” wound treatment (“SOC”; n=20), or treatment with a LLEC substrate as disclosed herein (n=18), were reviewed. The wound care device used in the present study consisted of a discrete matrix of silver and zinc dots. A sustained voltage of approximately 0.8 V was generated between the dots. The electric field generated at the device surface was measured to be 0.2-1.0 V, 10-50 μA.

Wounds were assessed until closed or healed. The number of days to wound closure and the rate of wound volume reduction were compared. Patients treated with LLEC substrate received one application of the device each week, or more frequently in the presence of excessive wound exudate, in conjunction with appropriate wound care management. The LLEC substrate was kept moist by saturating with normal saline or conductive hydrogel. Adjunctive therapies (such as negative pressure wound therapy [NPWT], etc.) were administered with SOC or with the use of the LLEC substrate unless contraindicated. The SOC group received the standard of care appropriate to the wound, for example antimicrobial dressings, barrier creams, alginates, silver dressings, absorptive dressings, hydrogel, enzymatic debridement ointment, NPWT, etc. Etiology-specific care was administered on a case-by-case basis. Dressings were applied at weekly intervals or more. The SOC and LLEC groups did not differ significantly in gender, age, wound types or the length, width, and area of their wounds.

Wound dimensions were recorded at the beginning of the treatment, as well as interim and final patient visits. Wound dimensions, including length (L), width (W) and depth (D) were measured, with depth measured at the deepest point. Wound closure progression was also documented through digital photography. Determining the area of the wound was performed using the length and width measurements of the wound surface area.

Closure was defined as 100% epithelialization with visible effacement of the wound. Wounds were assessed 1 week post-closure to ensure continued progress toward healing during its maturation and remodeling phase.

Wound types included in this study were diverse in etiology and dimensions, thus the time to heal for wounds was distributed over a wide range (9-124 days for SOC, and 3-44 days for the LLEC group). Additionally, the patients often had multiple co-morbidities, including diabetes, renal disease, and hypertension. The average number of days to wound closure was 36.25 (SD=28.89) for the SOC group and 19.78 (SD=14.45) for the LLEC group, p=0.036. On average, the wounds in the LLEC treatment group attained closure 45.43% earlier than those in the SOC group.

Based on the volume calculated, some wounds improved persistently while others first increased in size before improving. The SOC and the LLEC groups were compared to each other in terms of the number of instances when the dimensions of the patient wounds increased (i.e., wound treatment outcome degraded). In the SOC group, 10 wounds (50% for n=20) became larger during at least one measurement interval, whereas 3 wounds (16.7% for n=18) became larger in the LLEC group (p=0.018). Overall, wounds in both groups responded positively. Response to treatment was observed to be slower during the initial phase, but was observed to improve as time progressed.

The LLEC wound treatment group demonstrated on average a 45.4% faster closure rate as compared to the SOC group. Wounds receiving SOC were more likely to follow a “waxing-and-waning” progression in wound closure compared to wounds in the LLEC treatment group.

Compared to localized SOC treatments for wounds, the LLEC (1) reduces wound closure time, (2) has a steeper wound closure trajectory, and (3) has a more robust wound healing trend with fewer incidence of increased wound dimensions during the course of healing.

### Example 4

### LLEC Influence on Human Keratinocyte Migration

An LLEC-generated electrical field was mapped, leading to the observation that LLEC generates hydrogen peroxide, known to drive redox signaling. LLEC-induced phosphorylation of redox-sensitive IGF-1R was directly implicated in cell migration. The LLEC also increased keratinocyte mitochondrial membrane potential.

The LLEC substrate was made of polyester printed with dissimilar elemental metals. It comprises alternating circular regions of silver and zinc dots, along with a proprietary, biocompatible binder added to lock the electrodes to the surface of a flexible substrate in a pattern of discrete reservoirs. When the LLEC contacts an aqueous solution, the silver positive electrode (cathode) is reduced while the zinc negative electrode (anode) is oxidized. The LLEC used herein consisted of metals placed in proximity of about 1 mm to each other thus forming a redox couple and generating an ideal potential on the order of 1 Volt. The calculated values of the electric field from the LLEC were consistent with the magnitudes that are typically applied (1-10 V/cm) in classical electrotaxis experiments, suggesting that cell migration observed with the bioelectric dressing is likely due to electrotaxis.

Measurement of the potential difference between adjacent zinc and silver dots when the LLEC is in contact with de-ionized water yielded a value of about 0.2 Volts. Though the potential difference between zinc and silver dots can be measured, non-intrusive measurement of the electric field arising from contact between the LLEC and liquid medium was difficult. Keratinocyte migration was accelerated by exposure to an Ag/Zn LLEC. Replacing the Ag/Zn redox couple with Ag or Zn alone did not reproduce the effect of keratinocyte acceleration.

Exposing keratinocytes to an LLEC for 24 h significantly increased green fluorescence in the dichlorofluorescein (DCF) assay indicating generation of reactive oxygen species under the effect of the LLEC. To determine whether H₂O₂ is generated specifically, keratinocytes were cultured with a LLEC or placebo for 24 h and then loaded with PF6-AM (Peroxyfluor-6 acetoxymethyl ester; an indicator of endogenous H₂O₂). Greater intracellular fluorescence was observed in the LLEC keratinocytes compared to the cells grown with placebo. Over-expression of catalase (an enzyme that breaks down H₂O₂) attenuated the increased migration triggered by the LLEC. Treating keratinocytes with N-Acetyl Cysteine (which blocks oxidant-induced signaling) also failed to reproduce the increased migration observed with LLEC. Thus, H₂O₂ signaling mediated the increase of keratinocyte migration under the effect of the electrical stimulus.

External electrical stimulus can up-regulate the TCA (tricarboxylic acid) cycle. The stimulated TCA cycle is then expected to generate more NADH and FADH₂ to enter into the electron transport chain and elevate the mitochondrial membrane potential (Δm). Fluorescent dyes JC-1 and TMRM were used to measure mitochondrial membrane potential. JC-1 is a lipophilic dye which produces a red fluorescence with high Δm and green fluorescence when Δm is low. TMRM produces a red fluorescence proportional to Δm. Treatment of keratinocytes with LLEC for 24 h demonstrated significantly high red fluorescence with both JC-1 and TMRM, indicating an increase in mitochondrial membrane potential and energized mitochondria under the effect of the LLEC. As a potential consequence of a stimulated TCA cycle, available pyruvate (the primary substrate for the TCA cycle) is depleted resulting in an enhanced rate of glycolysis. This can lead to an increase in glucose uptake in order to push the glycolytic pathway forward. The rate of glucose uptake in HaCaT cells treated with LLEC was examined next. More than two fold enhancement of basal glucose uptake was observed after treatment with LLEC for 24 h as compared to placebo control.

Keratinocyte migration is known to involve phosphorylation of a number of receptor tyrosine kinases (RTKs). To determine which RTKs are activated as a result of LLEC, scratch assay was performed on keratinocytes treated with LLEC or placebo for 24 h. Samples were collected after 3 h and an antibody array that allows simultaneous assessment of the phosphorylation status of 42 RTKs was used to quantify RTK phosphorylation. It was determined that LLEC significantly induces IGF-1R phosphorylation. Sandwich ELISA using an antibody against phospho-IGF-1R and total IGF-1R verified this determination. As observed with the RTK array screening, potent induction in phosphorylation of IGF-1R was observed 3 h post scratch under the influence of LLEC. IGF-1R inhibitor attenuated the increased keratinocyte migration observed with LLEC treatment.

MBB (monobromobimane) alkylates thiol groups, displacing the bromine and adding a fluorescent tag (lamda emission=478 nm). MCB (monochlorobimane) reacts with only low molecular weight thiols such as glutathione. Fluorescence emission from UV laser-excited keratinocytes loaded with either MBB or MCB was determined for 30 min. Mean fluorescence collected from 10,000 cells showed a significant shift of MBB fluorescence emission from cells. No significant change in MCB fluorescence was observed, indicating a change in total protein thiol but not glutathione. HaCaT cells were treated with LLEC for 24 h followed by a scratch assay. Integrin expression was observed by immuno-cytochemistry at different time points. Higher integrin expression was observed 6 h post scratch at the migrating edge.

Consistent with evidence that cell migration requires H₂O₂ sensing, we determined that by blocking H₂O₂ signaling by decomposition of H₂O₂ by catalase or ROS scavenger, N-acetyl cysteine, the increase in LLEC-driven cell migration is prevented. The observation that the LLEC increases H₂O₂ production is significant because in addition to cell migration, hydrogen peroxide generated in the wound margin tissue is required to recruit neutrophils and other leukocytes to the wound, regulates monocyte function, and VEGF signaling pathway and tissue vascularization. Therefore, external electrical stimulation can be used as an effective strategy to deliver low levels of hydrogen peroxide over time to mimic the environment of the healing wound and thus should help improve wound outcomes. Another phenomenon observed during re-epithelialization is increased expression of the integrin subunit av. There is evidence that integrin, a major extracellular matrix receptor, polarizes in response to applied ES and thus controls directional cell migration. It may be noted that there are a number of integrin subunits, however we chose integrin αv because of evidence of association of αv integrin with IGF-1R, modulation of IGF-1 receptor signaling, and of driving keratinocyte locomotion. Additionally, integrin_{αv} has been reported to contain vicinal thiols that provide site for redox activation of function of these integrins and therefore the increase in protein thiols that we observe under the effect of ES may be the driving force behind increased integrin mediated cell migration. Other possible integrins which may be playing a role in LLEC-induced IGF-1R mediated keratinocyte migration are α5 integrin and α6 integrin.

Materials and Methods

Cell culture—Immortalized HaCaT human keratinocytes were grown in Dulbecco's low-glucose modified Eagle's medium (Life Technologies, Gaithersburg, Md., U.S.A.) supplemented with 10% fetal bovine serum, 100 U/ml penicillin, and 100 μg/ml streptomycin. The cells were maintained in a standard culture incubator with humidified air containing 5% CO₂ at 37° C.

Scratch assay—A cell migration assay was performed using culture inserts (IBIDI®, Verona, Wis.) according to the manufacturer's instructions. Cell migration was measured using time-lapse phase-contrast microscopy following withdrawal of the insert. Images were analyzed using the AxioVision Rel 4.8 software.

N-Acetyl Cysteine Treatment—Cells were pretreated with 5 mM of the thiol antioxidant N-acetylcysteine (Sigma) for 1 h before start of the scratch assay.

IGF-1R inhibition—When applicable, cells were preincubated with 50 nM IGF-1R inhibitor, picropodophyllin (Calbiochem, MA) just prior to the Scratch Assay.

Cellular H₂O₂ Analysis—To determine intracellular H₂O₂ levels, HaCaT cells were incubated with 5 pM PF6-AM in PBS for 20 min at room temperature. After loading, cells were washed twice to remove excess dye and visualized using a Zeiss Axiovert 200M microscope.

Catalase gene delivery—HaCaT cells were transfected with 2.3×10⁷ pfu AdCatalase or with the empty vector as control in 750 μL of media. Subsequently, 750 μL of additional media was added 4 h later and the cells were incubated for 72 h.

RTK Phosphorylation Assay—Human Phospho-Receptor Tyrosine Kinase phosphorylation was measured using Phospho-RTK Array kit (R & D Systems).

ELISA—Phosphorylated and total IGF-1R were measured using a DuoSet IC ELISA kit from R&D Systems.

Determination of Mitochondrial Membrane Potential—Mitochondrial membrane potential was measured in HaCaT cells exposed to the LLEC or placebo using TMRM or JC-1 (MitoProbe JC-1 Assay Kit for Flow Cytometry, Life Technologies), per manufacturer's instructions for flow cytometry.

Integrin αV Expression—Human HaCaT cells were grown under the MCD or placebo and harvested 6 h after removing the IBIDI® insert. Staining was done using antibody against integrin αV (Abcam, Cambridge, Mass.).

### Example 5

### Generation of Superoxide

A LLEC substrate was tested to determine the effects on superoxide levels which can activate signal pathways. PROCELLERA® LLEC substrate increased cellular protein sulfhydryl levels. Further, the PROCELLERA® substrate increased cellular glucose uptake in human keratinocytes. Increased glucose uptake can result in greater mitochondrial activity and thus increased glucose utilization, providing more energy for cellular migration and proliferation. This can speed wound healing.

### Example 6

### Effect on Propionibacterium acnes

Bacterial Strains and Culture

The main bacterial strain used in this study is Propionibacterium acnes and multiple antibiotics-resistant P. acnes isolates are to be evaluated.

ATCC medium (7 Actinomyces broth) (BD) and/or ATCC medium (593 chopped meat medium) is used for culturing P. acnes under an anaerobic condition at 37° C. All experiments are performed under anaerobic conditions.

Culture

LNA (Leeming-Notman agar) medium is prepared and cultured at 34° C. for 14 days.

Planktonic Cells

P. acnes is a relatively slow-growing, typically aero-tolerant anaerobic, Gram-positive bacterium (rod). P. acnes is cultured under anaerobic condition to determine for efficacy of an embodiment disclosed herein (PROCELLERA®). Overnight bacterial cultures are diluted with fresh culture medium supplemented with 0.1% sodium thioglycolate in PBS to 10⁵ colony forming units (CFUs). Next, the bacterial suspensions (0.5 mL of about 105) are applied directly on PROCELLERA® (2″×2″) and control fabrics in Petri-dishes under anaerobic conditions. After 0 h and 24 h post treatments at 37° C., portions of the sample fabrics are placed into anaerobic diluents and vigorously shaken by vortexing for 2 min. The suspensions are diluted serially and plated onto anaerobic plates under an anaerobic condition. After 24 h incubation, the surviving colonies are counted. The LLEC limits bacterial proliferation.

### Example 7

### Post-Treatment—Surgical Procedures

After surgery to the abdomen, the doctor applies a disclosed resorbable device within the incision site prior to closing the wound. The device will aid in healing as well as prevent or minimize bacterial growth.

### Example 8

### Post-Treatment—Surgical Procedures

After surgery to the leg, the doctor applies a disclosed resorbable device within the incision site prior to closing the wound. The device will aid in healing as well as prevent or minimize bacterial growth. The device also comprises means for temperature monitoring that can transmit this data to a primary caregiver.

### Example 9

### Post-Treatment—Surgical Procedures

After surgery to the back, the doctor applies a disclosed resorbable device within the incision site prior to closing the wound. The device will aid in healing as well as prevent or minimize bacterial growth. The device also comprises an accelerometer that can transmit collected movement data to a primary caregiver or data collection device or system.

### Example 10

### Resorbable Pouch

To prepare a resorbable pouch, 18.0 g of polymer, 1.0 g minocycline and 1.0 g rifampin are dissolved in 75 mL of a solution of tetrahydrofuran-methanol. This solution is poured over a level non-stick Teflon surface. A calibrated stainless steel gardner knife is used to spread the solution to the desired thickness, typically to a range of from about 2 and about 400 microns.

The film is dried at ambient temperature overnight. Thereafter, the solvent cast film is dried in a convection oven at 50° C. for 1 day, the temperature of the oven is increased to 80° C., and the film is dried further for 2 days. At this point, the dried film is ready for laser cutting and further processing to create the pouch, i.e., cutting and sealing three of the sides. The pouch is cut for overall shape to match the desired CRM and to create a mesh like covering.

### Example 11

### Resorbable Pouch

To prepare a resorbable clamshell pouch, a film is prepared as described above in Example 10. After the film is dried, it is thermoformed into the clamshell shape by placing a film sheet into a frame and heating the film to about 90-100° C. and lowering the frame over a clamshell-shaped mold whereby the film takes the shape of the clamshell mold. The molded clamshell is cooled, freed from the mold, and laser cut from the film sheet.

### Example 12

### Breast Augmentation

The patient is prepared for surgery, including the surgeon using a marker to indicate where the breast implant incisions will be made. The incision location is pre-treated with a multi-array matrix as disclosed herein.

The patient is scrubbed with an antimicrobial soap to minimize the risk of infection, and then hooked up to several monitors so that the surgical team can monitor blood pressure, heart rate and other vital signs. The legs are placed in inflatable plastic compression sleeves, which inflate and deflate periodically during the surgery to help prevent deep venous thrombosis (DVT), or the formation of blood clots deep in the veins of the legs.

An IV is inserted on the inside of the elbow to keep the patient hydrated and also administer medications, including sedatives and anesthesia. There are several choices for anesthesia during breast implant surgery. These include general anesthesia, conscious sedation and local anesthesia.

Next, the surgeon makes the breast implant incisions. She creates a pocket behind the breast tissue or under the pectoral muscle tissue based on the implant placement position chosen during your consultation. Once the pocket is created, the breast implant as described herein comprising a multi-array matrix on its surface shell is inserted. If the patient opted for silicone-filled implants, the already-filled implants will be put in place; saline breast implants are usually filled after the shell is put in the pocket.

If the patient has small breasts, or is having implants inserted after a mastectomy (surgical removal of one or both breasts), the patient may need a tissue expander comprising a multi-array matrix on its surface shell inserted for a period of time before receiving the permanent breast implant(s). A tissue expander looks like a regular saline implant, but it has a port through which additional saline can be added. This allows the surgeon to stretch the breast and make room for the implant.

Expandable saline breast implants work in a similar manner to tissue expanders, but are permanent. A filler port is left near the incision, and more saline is added every week until the patient is satisfied. At that time, the filler port is removed and the saline implant seals itself shut. More saline can be added for up to six months after surgery.

After the breast implants are in place, the surgeon will check for symmetry. She will place the patient in an upright position to see how the implants look when seated. If everything looks good, your surgeon will close the incisions. A dressing comprising a multi-array matrix on its surface will be applied to protect the wounds, keep the tissue and implant securely in place, and to reduce swelling.

In closing, it is to be understood that although aspects of the present specification are highlighted by referring to specific embodiments, one skilled in the art will readily appreciate that these disclosed embodiments are only illustrative of the principles of the subject matter disclosed herein. Therefore, it should be understood that the disclosed subject matter is in no way limited to a particular methodology, protocol, and/or reagent, etc., described herein. As such, various modifications or changes to or alternative configurations of the disclosed subject matter can be made in accordance with the teachings herein without departing from the spirit of the present specification. Lastly, the terminology used herein is for the purpose of describing particular embodiments only, and is not intended to limit the scope of the present disclosure, which is defined solely by the claims. Accordingly, embodiments of the present disclosure are not limited to those precisely as shown and described.

Certain embodiments are described herein, comprising the best mode known to the inventor for carrying out the methods and devices described herein. Of course, variations on these described embodiments will become apparent to those of ordinary skill in the art upon reading the foregoing description. Accordingly, this disclosure comprises all modifications and equivalents of the subject matter recited in the claims appended hereto as permitted by applicable law. Moreover, any combination of the above-described embodiments in all possible variations thereof is encompassed by the disclosure unless otherwise indicated herein or otherwise clearly contradicted by context.

Groupings of alternative embodiments, elements, or steps of the present disclosure are not to be construed as limitations. Each group member may be referred to and claimed individually or in any combination with other group members disclosed herein. It is anticipated that one or more members of a group may be comprised in, or deleted from, a group for reasons of convenience and/or patentability. When any such inclusion or deletion occurs, the specification is deemed to contain the group as modified thus fulfilling the written description of all Markush groups used in the appended claims.

Unless otherwise indicated, all numbers expressing a characteristic, item, quantity, parameter, property, term, and so forth used in the present specification and claims are to be understood as being modified in all instances by the term “about.” As used herein, the term “about” means that the characteristic, item, quantity, parameter, property, or term so qualified encompasses a range of plus or minus ten percent above and below the value of the stated characteristic, item, quantity, parameter, property, or term. Accordingly, unless indicated to the contrary, the numerical parameters set forth in the specification and attached claims are approximations that may vary. At the very least, and not as an attempt to limit the application of the doctrine of equivalents to the scope of the claims, each numerical indication should at least be construed in light of the number of reported significant digits and by applying ordinary rounding techniques. Notwithstanding that the numerical ranges and values setting forth the broad scope of the disclosure are approximations, the numerical ranges and values set forth in the specific examples are reported as precisely as possible. Any numerical range or value, however, inherently contains certain errors necessarily resulting from the standard deviation found in their respective testing measurements. Recitation of numerical ranges of values herein is merely intended to serve as a shorthand method of referring individually to each separate numerical value falling within the range. Unless otherwise indicated herein, each individual value of a numerical range is incorporated into the present specification as if it were individually recited herein.

The terms “a,” “an,” “the” and similar referents used in the context of describing the disclosure (especially in the context of the following claims) are to be construed to cover both the singular and the plural, unless otherwise indicated herein or clearly contradicted by context. All methods described herein can be performed in any suitable order unless otherwise indicated herein or otherwise clearly contradicted by context. The use of any and all examples, or exemplary language (e.g., “such as”) provided herein is intended merely to better illuminate the disclosure and does not pose a limitation on the scope otherwise claimed. No language in the present specification should be construed as indicating any non-claimed element essential to the practice of embodiments disclosed herein.

Specific embodiments disclosed herein may be further limited in the claims using consisting of or consisting essentially of language. When used in the claims, whether as filed or added per amendment, the transition term “consisting of” excludes any element, step, or ingredient not specified in the claims. The transition term “consisting essentially of” limits the scope of a claim to the specified materials or steps and those that do not materially affect the basic and novel characteristic(s). Embodiments of the present disclosure so claimed are inherently or expressly described and enabled herein.

1. A resorbable device comprising a substrate comprising one or more biocompatible electrodes configured to generate at least one of a low level electric field (LLEF) or low level electric current (LLEC).

2. The device of claim 1 wherein the biocompatible electrodes comprise a first array comprising a pattern of microcells formed from a first conductive material, and a second array comprising a pattern of microcells formed from a second conductive material.

3. The device of claim 2 wherein the first conductive material and the second conductive material comprise the same material.

4. The device of claim 3 wherein the first array and second array each comprise a discrete circuit.

5. The device of claim 4, further comprising a power source.

6. The device of claim 2 wherein the first array and the second array spontaneously generate a LLEF.

7. The device of claim 6 wherein the first array and the second array spontaneously generate a LLEC when contacted with an electrolytic solution.

8. The device of claim 7, wherein said device is in a pouch configuration.

9. The device of claim 7, wherein said device is in a tape configuration.

10. A method for treating an injury comprising applying the device of claim 7 to the injury.

11. The method of claim 10 wherein said injury comprises a surgical incision.

12. The method of claim 11 wherein said surgical incision comprises an abdominal incision.

13. The method of claim 12 wherein said surgical incision comprises an upper abdominal incision.

14. The method of claim 10 wherein said injury comprises a traumatic injury.

15. A breast implant comprising one or more biocompatible electrodes configured to generate at least one of a low level electric field (LLEF) or low level electric current (LLEC).

16. The implant of claim 15 wherein the biocompatible electrodes comprise a first array comprising a pattern of microcells formed from a first conductive material, and a second array comprising a pattern of microcells formed from a second conductive material.

17. The implant of claim 16 wherein the first conductive material and the second conductive material comprise the same material.

18. The implant of claim 17 wherein the first array and second array each comprise a discrete circuit.

19. The implant of claim 18, further comprising a power source.

20. The device of claim 16 wherein the first array and the second array spontaneously generate a LLEF.

21. The implant of claim 20 wherein the first array and the second array spontaneously generate a LLEC when contacted with an electrolytic solution.

22. A method for performing breast augmentation or breast reconstruction surgery, said method comprising insertion of a breast implant comprising one or more biocompatible electrodes configured to generate at least one of a low level electric field (LLEF) or low level electric current (LLEC).

23. The method of claim 22 wherein said surgery comprises a surgical incision.

24. The method of claim 23 wherein said surgical incision comprises an abdominal incision.

25. The method of claim 24 wherein said surgical incision comprises at least one of an Inframammary incision, a periareolar incision, a transaxillary incision, a transumbilical incision, a transabdominal incision, or combinations thereof.

26. The method of claim 22 wherein said surgery comprises a subglandular, subfacial, or subpectoral emplacement of said breast implant.
